(19) United States
(12) Patent Application Publication
(10) Pub. No.: US 2016/0120858 A1
(43) Pub. Date: May 5, 2016

(54) PHARMACEUTICAL COMPOSITION CONTAINING CLIOQUINOL FOR TREATING AUTISM SPECTRUM DISORDERS

(71) Applicants: ACADEMIA SINICA, Taipei (TW); INSTITUTE FOR BASIC SCIENCE, Daejeon (KR); KOREA ADVANCED INSTITUTE OF SCIENCE AND TECHNOLOGY, Daejeon (KR); THE ASAN FOUNDATION, Seoul (KR)

(72) Inventors: Eun-Joon KIM, Daejeon (KR); Eun-Jae LEE, Seoul (KR); Hye-Jin LEE, Daejeon (KR); Yi-Ping HSUEH, Taipei (TW); Tzyy-Nan Huang, Taipei (TW); Jae-Young KOH, Seoul (KR)

(21) Appl. No.: 14/932,812

(22) Filed: Nov. 4, 2015

(30) Foreign Application Priority Data
Nov. 5, 2014 (KR) 10-2014-0152903

(51) Int. Cl. A61K 31/47; A61K 9/00

(52) U.S. Cl. CPC A61K 31/47; A61K 9/0019; A23L 1/30; A23V 2002/00

(57) ABSTRACT

The present invention relates to a pharmaceutical composition for treating autism spectrum disorders, which includes clioquinol or pharmaceutically acceptable salt thereof. In addition, the present invention relates to a food composition for treating autism spectrum disorders, which includes clioquinol or pharmaceutically acceptable salt thereof.

### CROSS-REFERENCE TO RELATED APPLICATION

This application claims the benefit of Korean Patent

Application No. 10-2014-0152903, filed on Nov. 5, 2014 in the Korean Intellectual Property Office, the entirety of which is incorporated herein by reference.

### BACKGROUND OF THE INVENTION

1. Field of the Invention

The present invention relates to a pharmaceutical composition for treating autism spectrum disorders and a food composition for improving autism spectrum disorders.

2. Description of the Related Art

Autism spectrum disorder (ASD) indicates neural development disorders characterized by social interaction and communication disorders, limited and repetitive behaviors, attentions and actions. ASDs affect ˜1% of the population and are thought to be strongly influenced by genetic factors. A large number of ASD-associated genetic variations have recently been identified, indicating that ASDs represent a genetically heterogeneous family of disorders. Some of the genetic variations follow common pathways/functions, including synaptic transmission, transcriptional regulation, and chromatin remodeling. In addition, in the research using the mouse model of ASD having this mutation, possible mechanisms underlying the occurrence of ASD, in other words, glutamate impairment, and imbalance between suppression synapses have been proposed.

Environmental influences, such as nutrition, toxins and poisons, drugs, infection and stress, are thought to have a significant influence on psychiatric disorders. In ASDs, well-known examples of environmental influences include pre- or perinatal exposure to viruses or teratogens such as valproic acid and thalidomide. However, studies on additional environmental influences and underlying mechanisms are at an early stage. This contrasts with the rapidly growing evidence for the contribution of genetic factors to ASDs. Because environmental factors are highly likely to interact with the genetic variations of ASD to determine the type, severity, and trajectory of ASD symptoms, a balance between genetic and environmental causes is required in studies of ASDs.

Zinc (Zn), the second-most abundant trace element with a critical role in human nutrition and health, regulates a variety of cellular processes and protein functions. Zn deficiency has been implicated in diverse neurological and psychiatric disorders, including Alzheimer's disease, Parkinson's disease, attention deficit/hyperactivity disorder, schizophrenia, epilepsy, and mood disorders. The association of Zn with ASDs has been suggested based on its deficiency in individuals with ASDs, including a recent large cohort of children, as well as the phenotypes of Zn-deficient experimental animals. This association is further supported by the potential therapeutic value of Zn supplementation in ASD treatment. However, strong evidence supporting the association between Zn deficiency and ASDs is largely unavailable, and the mechanisms underlying the association remain obscure.

In the synapse, the main pool of Zn ions is presynaptic vesicles where Zn is in the millimolar range, whereas postsynaptic sites contain much smaller amounts of Zn (picomolar range). Presynaptic free Zn is co-released with glutamate during neuronal activity and serves to suppress NMDA receptors (NMDARs) in the synaptic cleft. Some Zn ions enter the postsynaptic sites through calcium channels, NMDA receptors, and calcium-permeable AMPA receptors (AMPARs), and regulate target proteins such as NMDARs and TrkB receptors, through mechanisms including those involving Src family kinases (SFKs). Another important effector of postsynaptic Zn is Shank (also known as ProSAP), a family of excitatory postsynaptic scaffolding proteins with three known members (Shank1/2/3). Zn binds to Shank2/3 and enhances their postsynaptic stabilization, promoting excitatory synapse formation and maturation.

Shank 2/3 (also known as ProSAP1/2), a member of the Shank family of postsynaptic scaffolding proteins, is associated with ADS through human gene researched and mouse model/incubation neuron researches. Mice having the Shank2/3 mutations show various impairments in the glutamate synapses. One notable change is the reduction in NMDAR function observed in Shank2^{-/-} mice (exons 6+7 deletion).

Meanwhile, clioquinol (5-Chloro-7-Iodo-8-Hydroxyquinoline) is a compound belonging to Hydroxy-quinoline.

Clioquinol is known for usages in amoebiasis medication, contagious diarrhea medication, anti-obesity medication, Alzheimer's disease medication, etc. (WO1998/0764030).

Inventors of the present invention completed the present invention by identifying that the behaviors due to the autism spectrum disorder in the model mice of the autism spectrum disorder are improved by administrating clioquinol while researching the autism spectrum disorder.

### SUMMARY OF THE INVENTION

An object of the present invention is to provide a pharmaceutical composition for treating autism spectrum disorders.

Another object of the present invention is to provide a food composition for treating autism spectrum disorders.

To achieve the object, the present invention provides a pharmaceutical composition for treating autism spectrum disorders, which includes clioquinol or pharmaceutically acceptable salt thereof.

In addition, the present invention provides a food composition for treating autism spectrum disorders, which includes clioquinol or pharmaceutically acceptable salt.

The composition of the present invention treats and improves autism spectrum disorders.

### BRIEF DESCRIPTION OF THE DRAWINGS

FIGS. 1 to 7 show that clioquinol (CQ) sharply improves social interactions in Shank2^{-/-} mice.

FIGS. 1 to 3

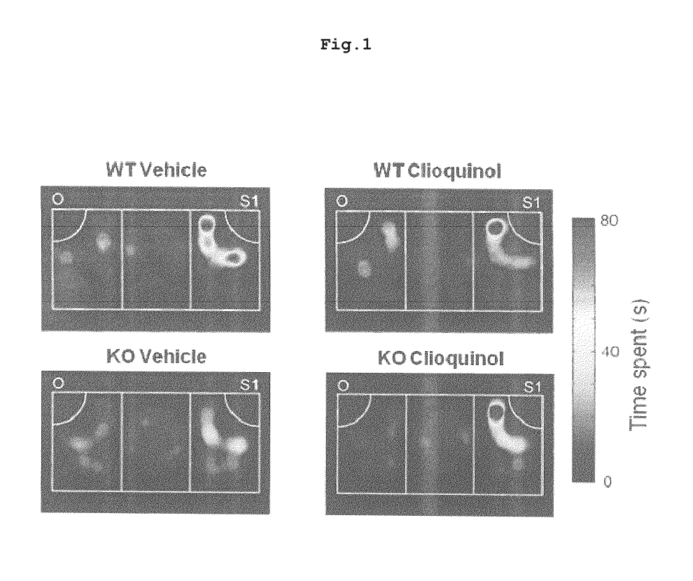

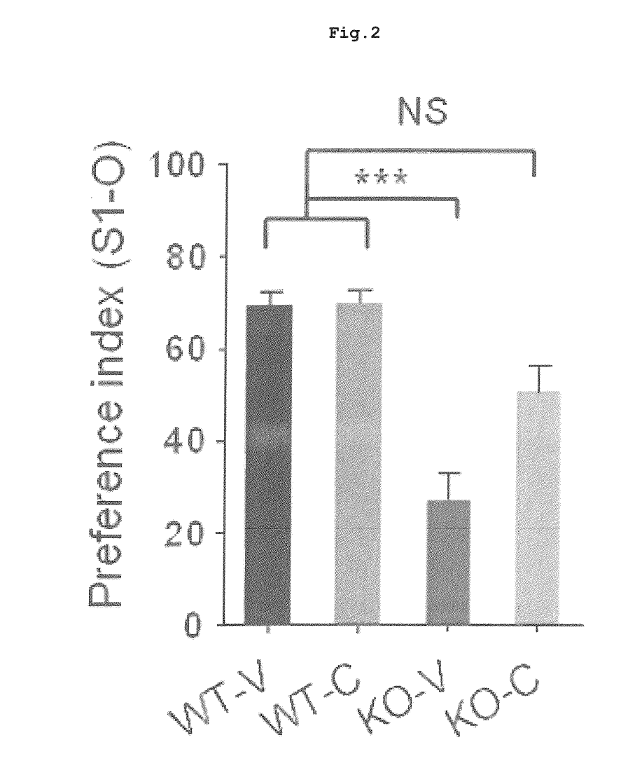

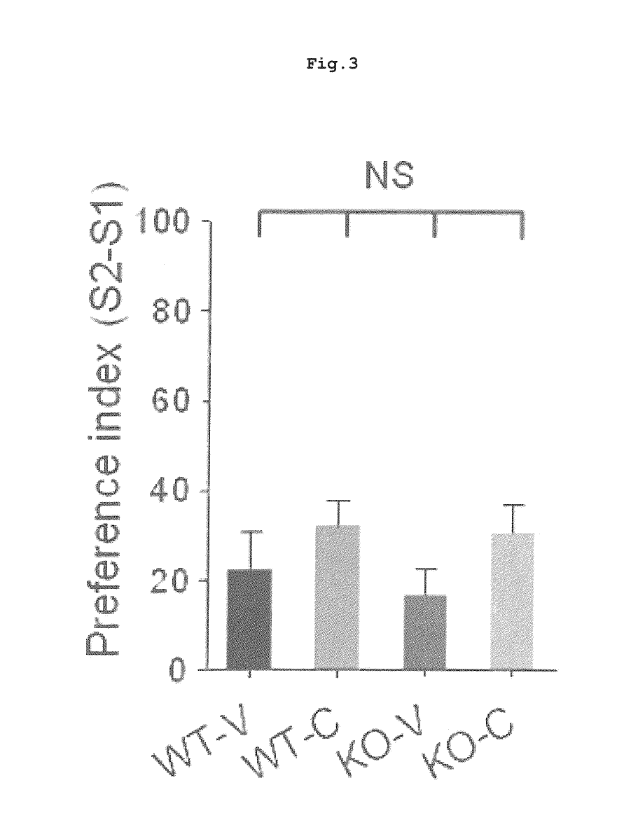

Clioquinol improves social interaction in Shank2^{-/-} (KO) mice, however, has no effect on wild-type (WT) mice (FIGS. 1 and 2). Social novelty recognition level is similar in wild-type (WT) and shank2^{-/-} mice, and clioquinol (CQ) did not affect the social novelty recognition of these mice (FIG. 3). Mice were injected with clioquinol (30 mg/kg; i.p.), or vehicle, 2 hours prior to behavioral tests. The heat map of FIG. 1 shows an example of the movement of mice. The social preference index represents the numerical difference between the times spent exploring the two targets (S1/stranger vs. O/object, or S2/new stranger vs. S1/previous stranger) divided by total time spent×100. (n=18 for wild-type, vehicle (WT-V) and wild-type, clioquinol (WT-C), n=16 for knockout, vehicle (KO-V) and knockout, clioquinol (KO-C), NS indicates “not significant”, ***p<0.001, Kruskal-Wallis one-way analysis of variance (ANOVA) with Dunn's post hoc test).

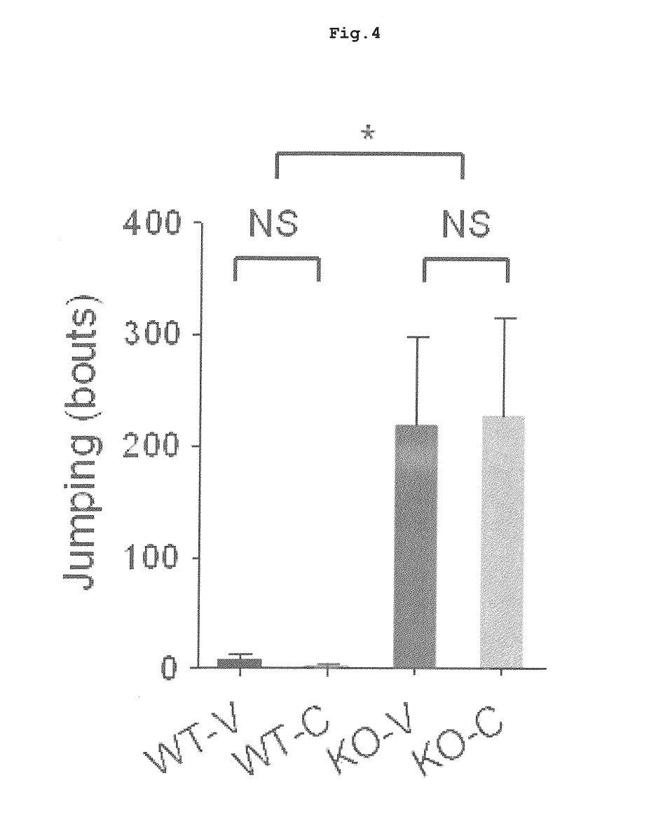

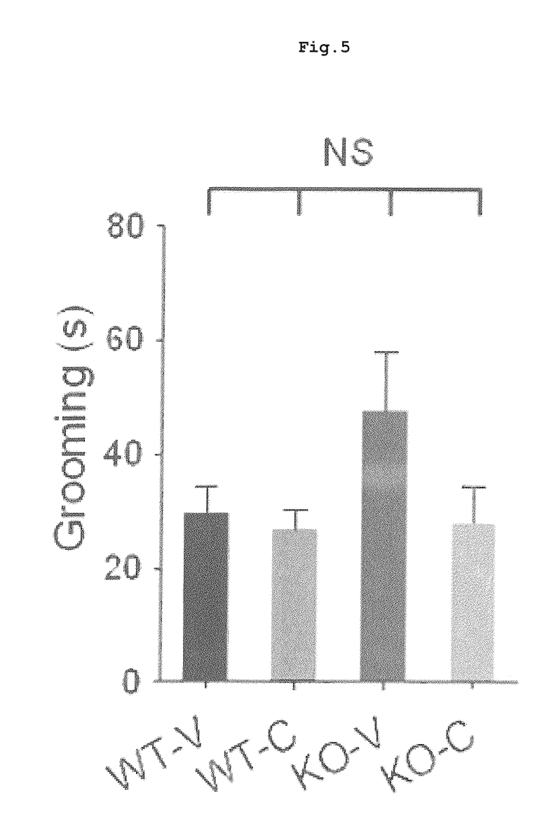

FIGS. 4 and 5

Clioquinol (CQ) had no effect on jumping or grooming in the shank2^{-/-} mice. (n=10 for WT-V and WT-C, n=11 for KO-V and KO-C, *p<0.05, two-way ANOVA and Kruskal-Wallis one-way ANOVA with Dunn's post hoc test). Data are presented as mean±SEM.

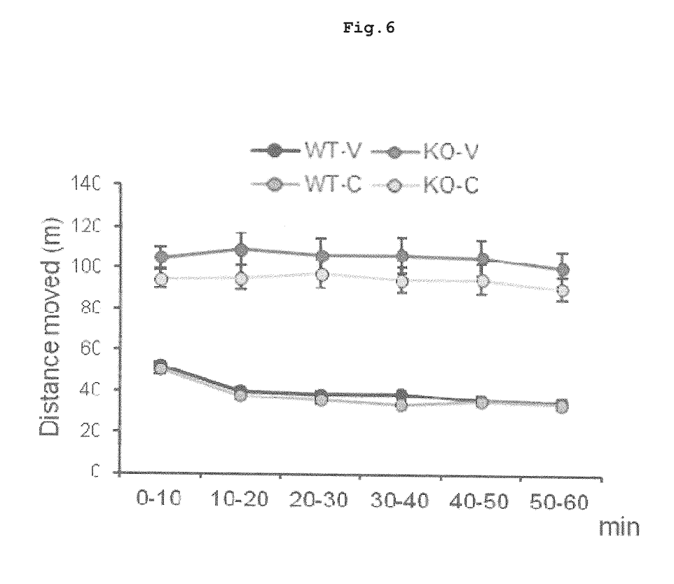

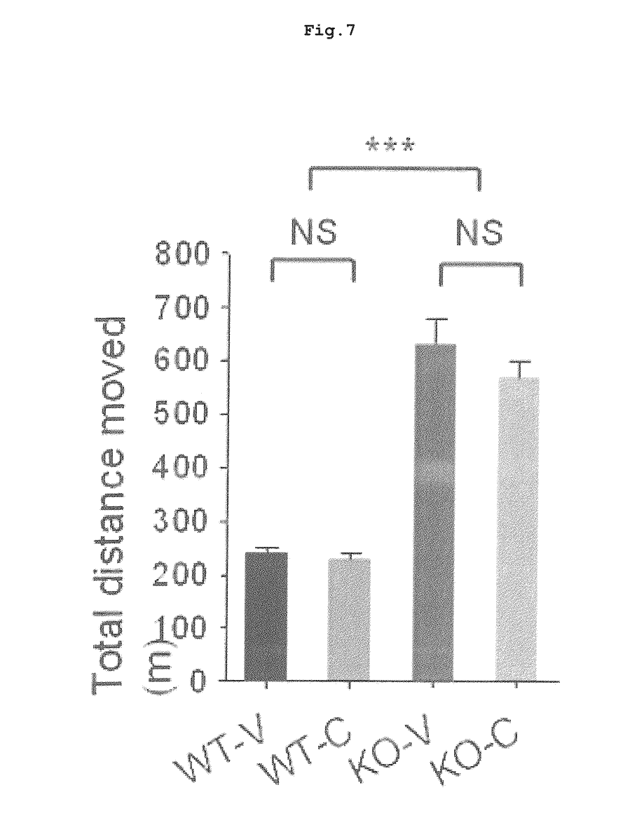

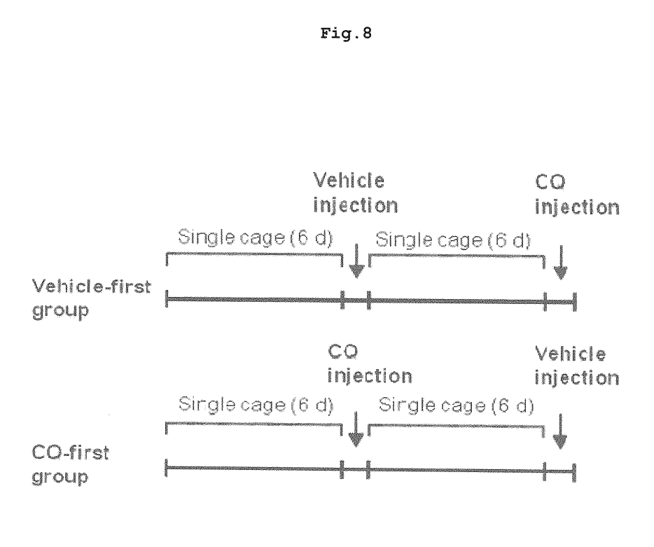

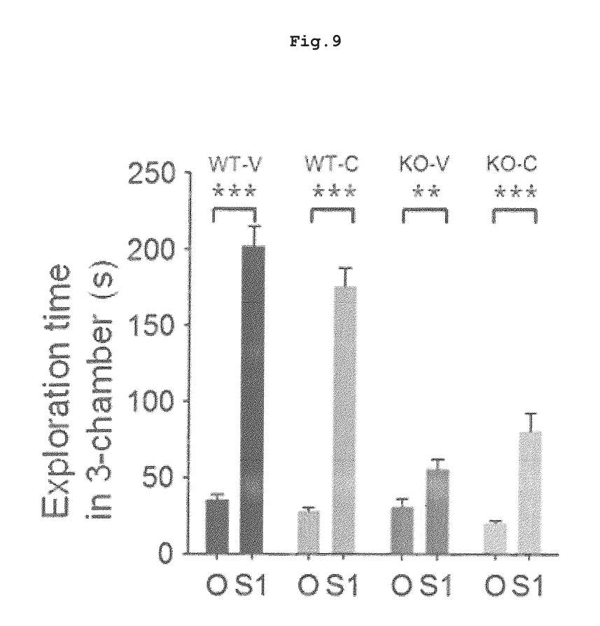

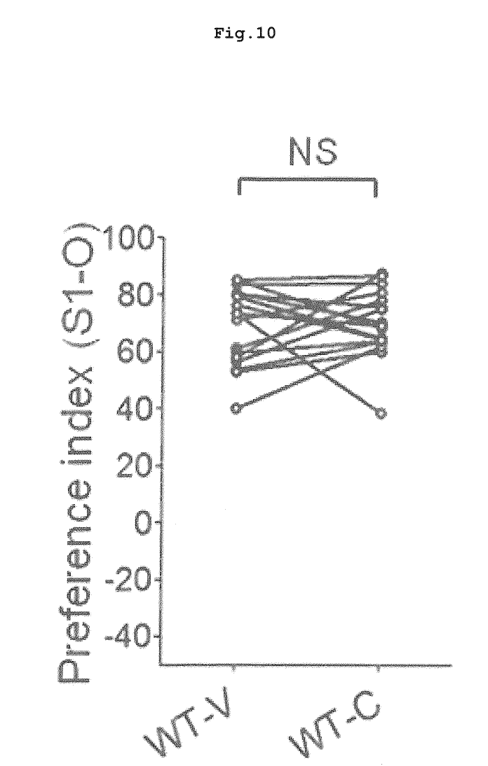

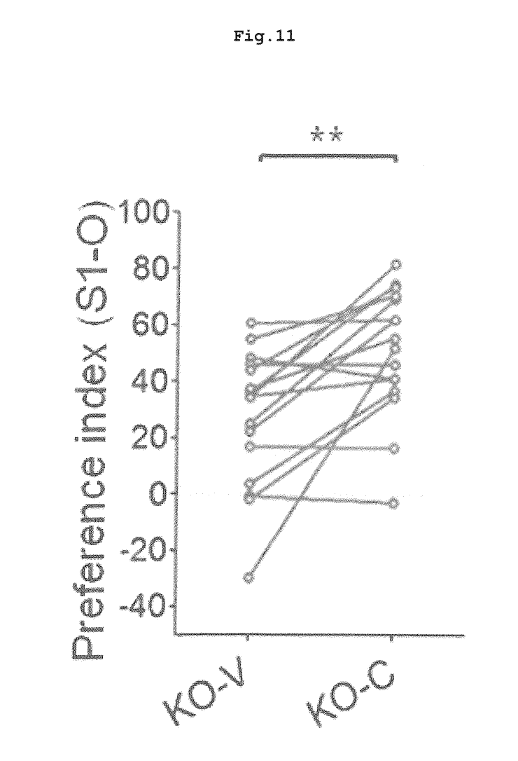

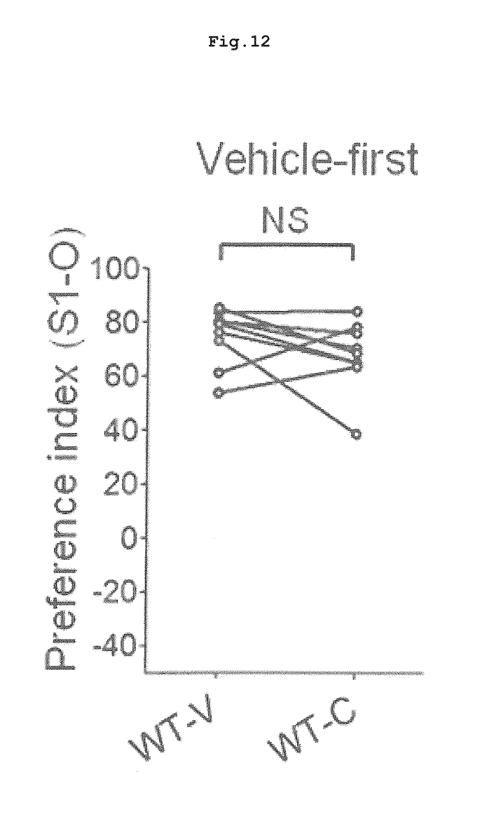

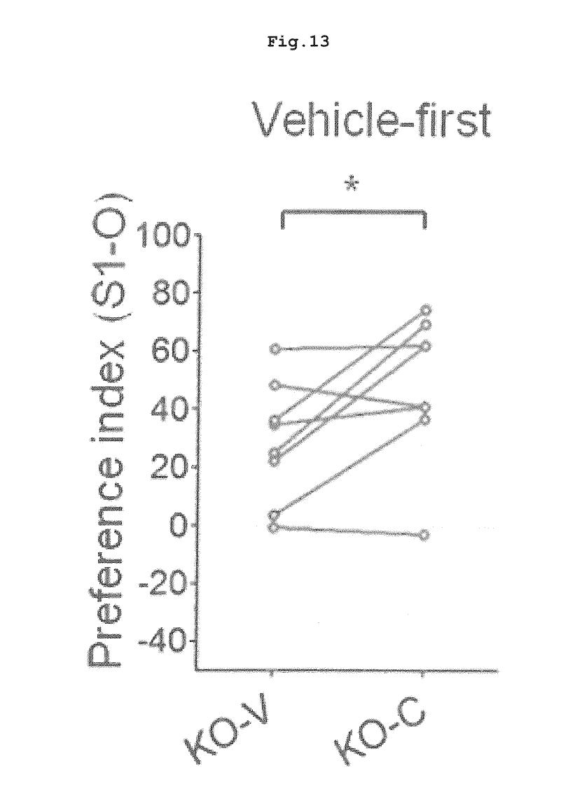

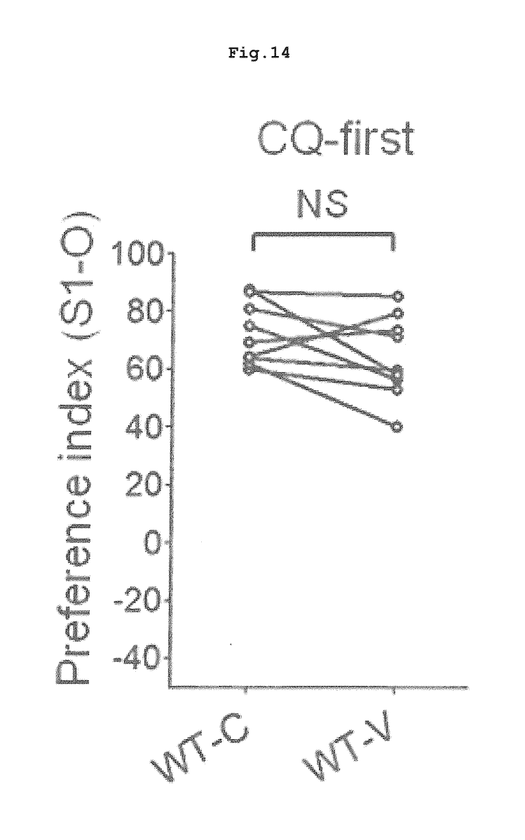

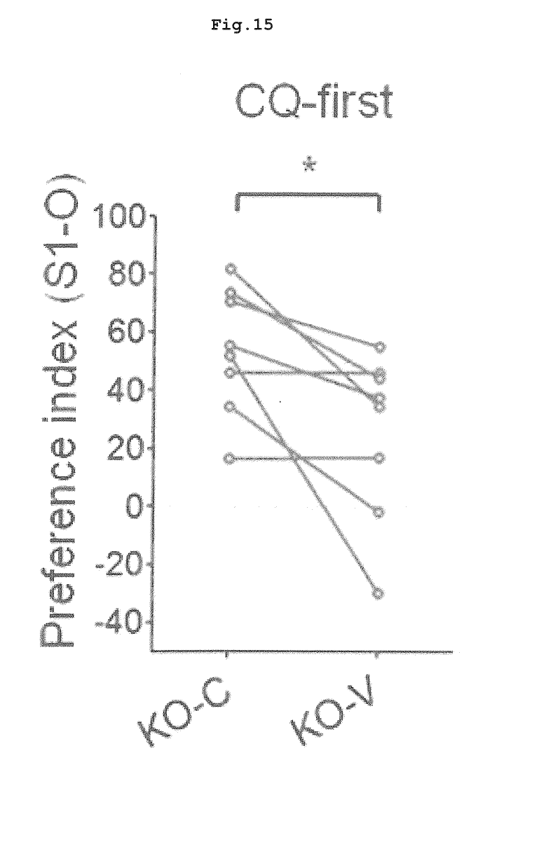

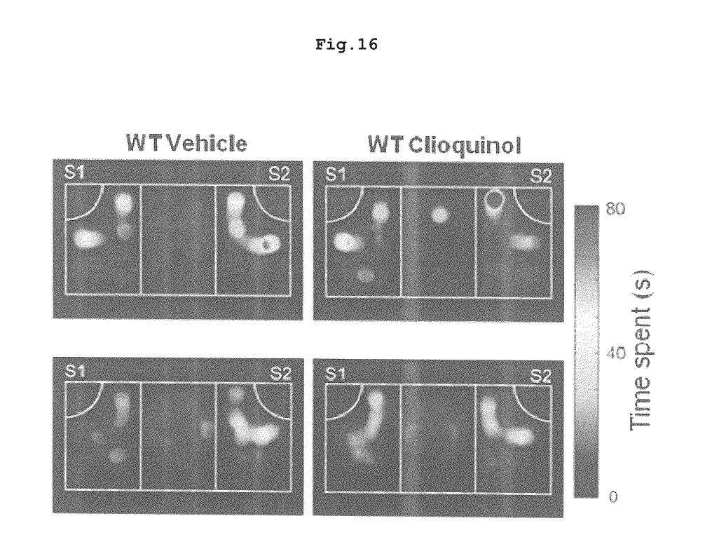

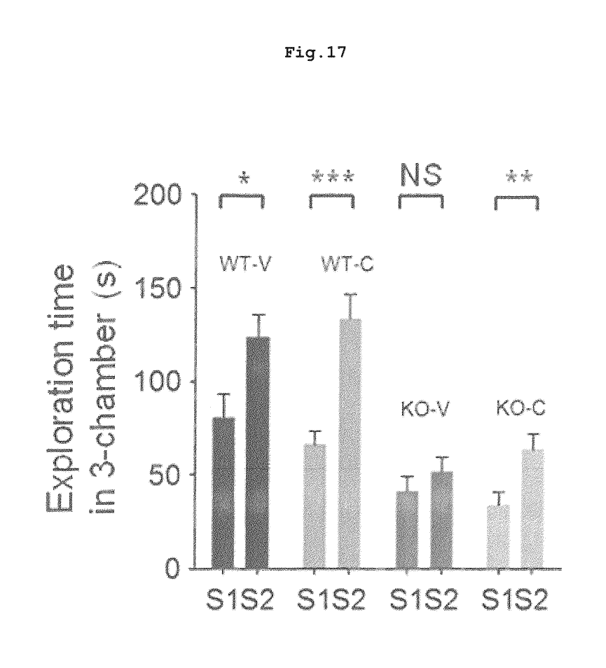

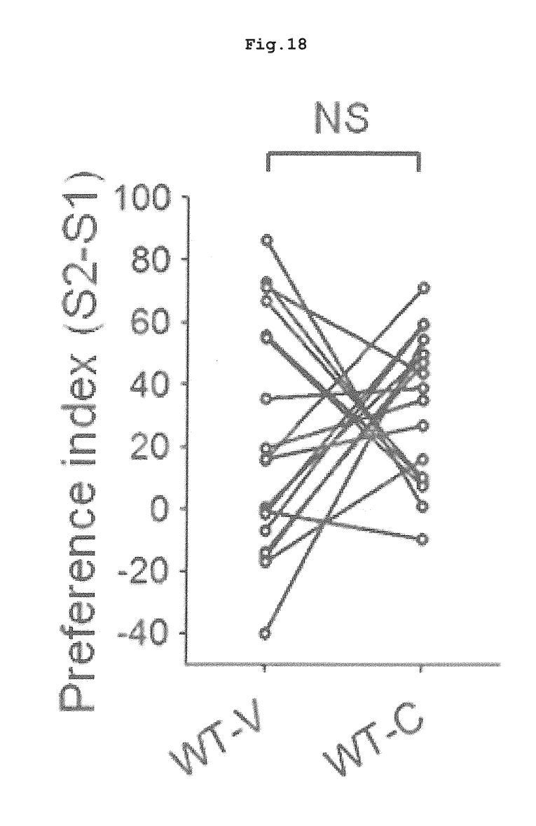

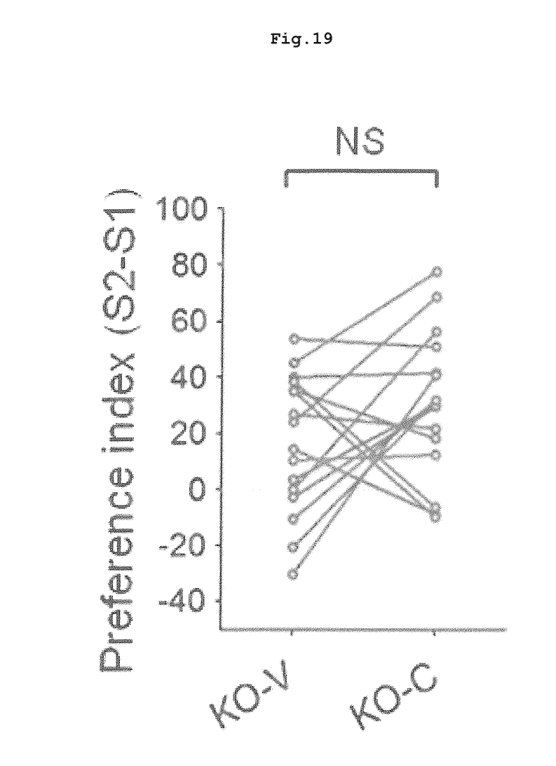

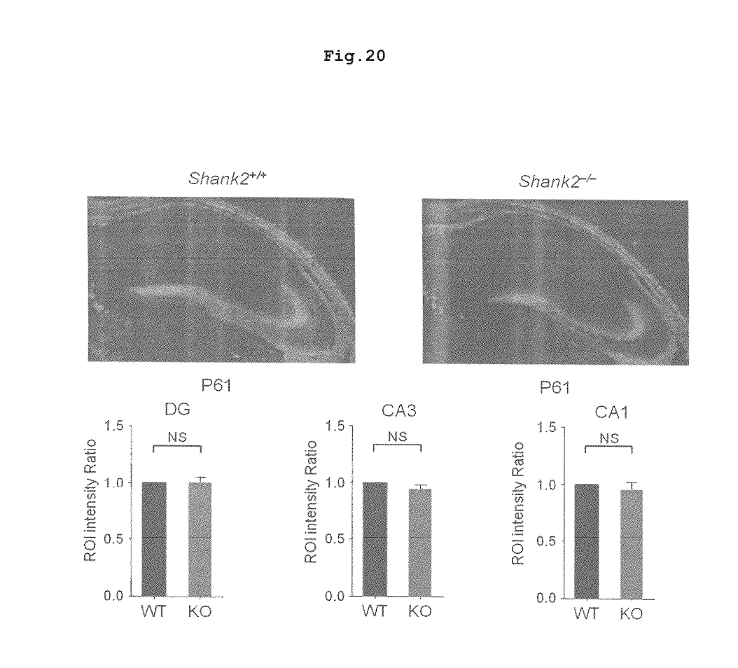

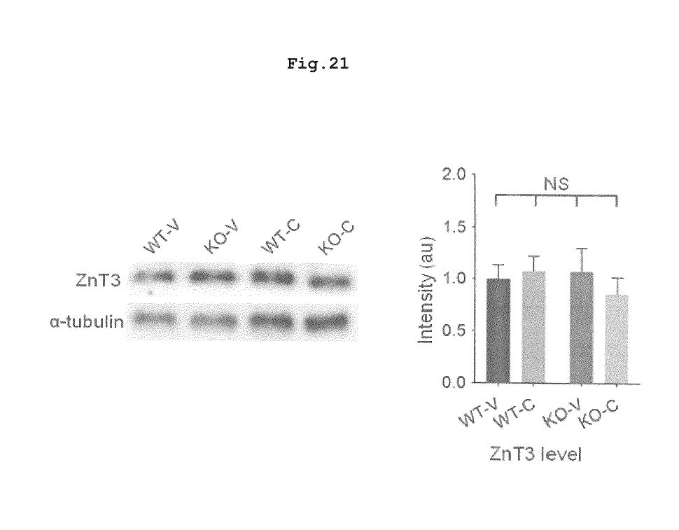

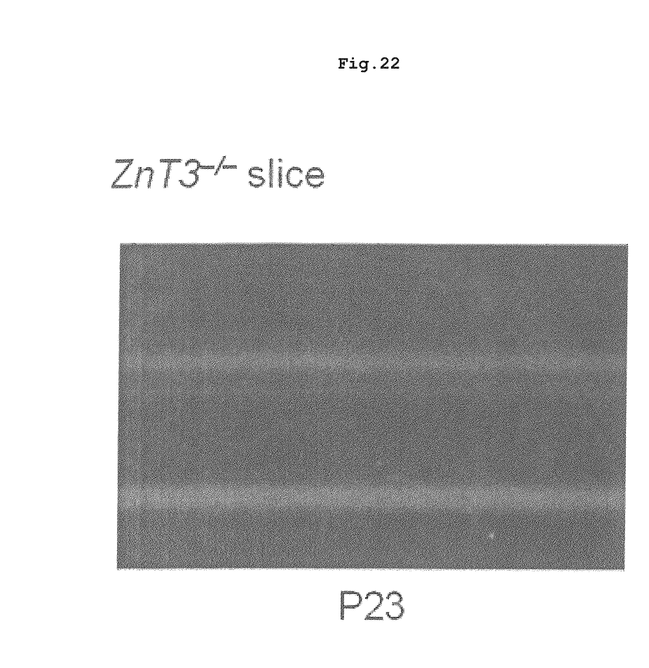

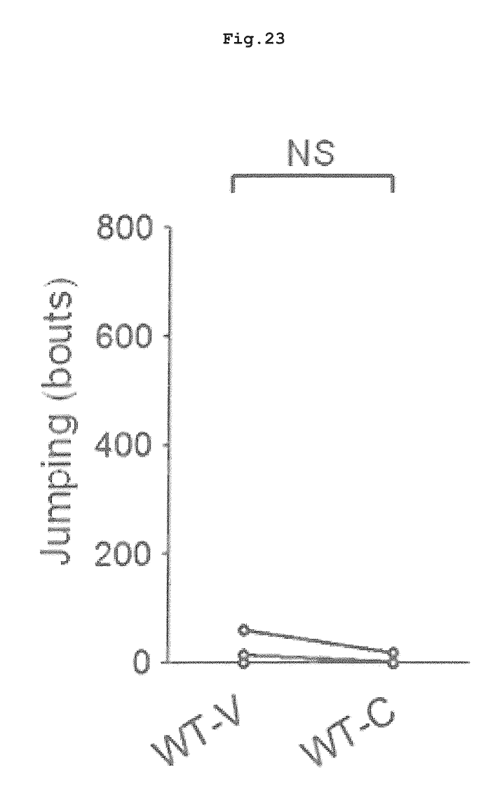

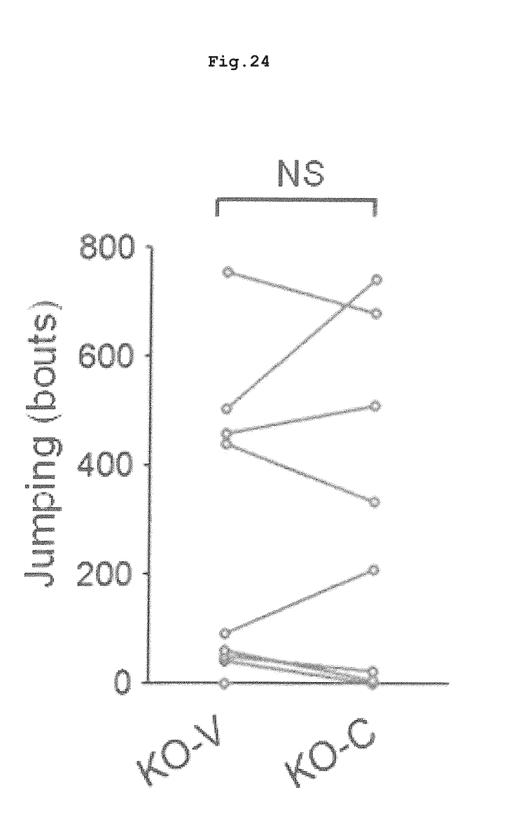

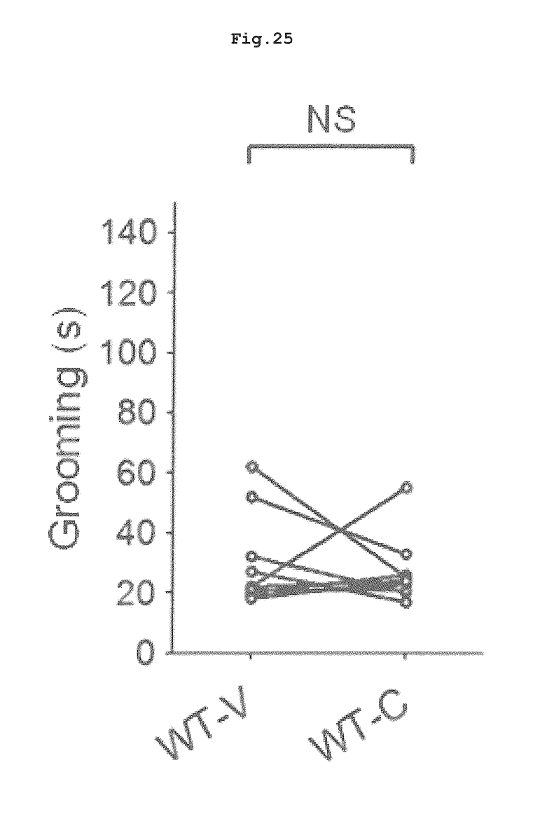

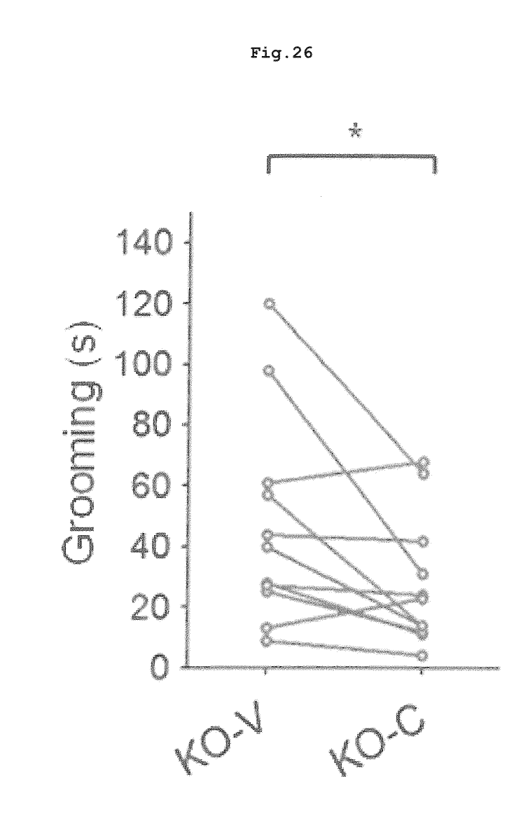

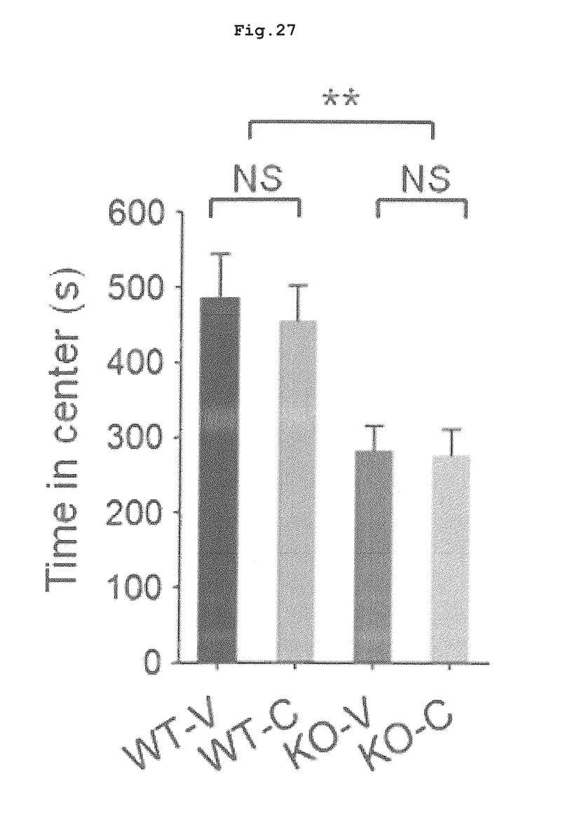

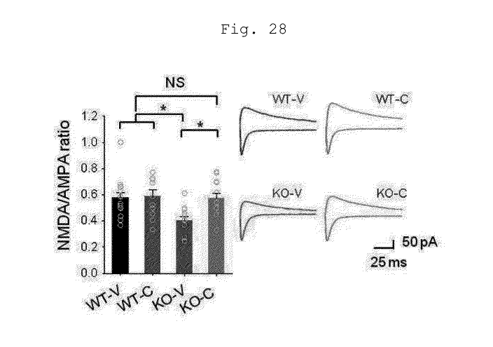

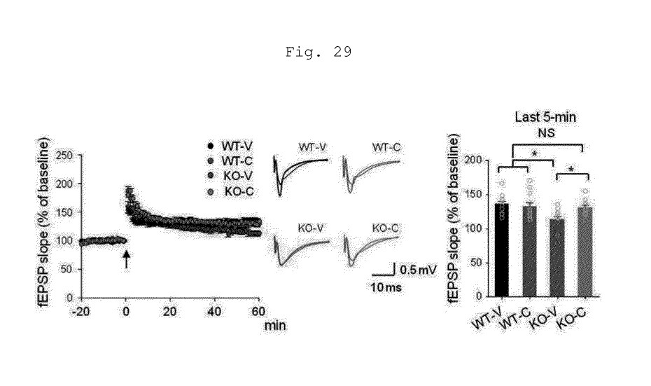

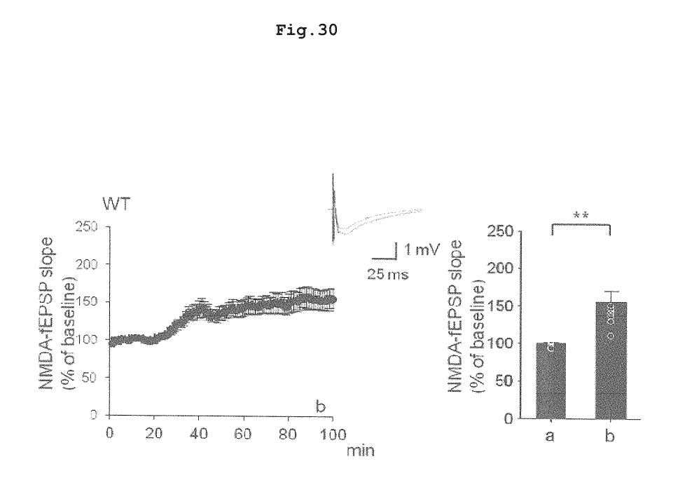

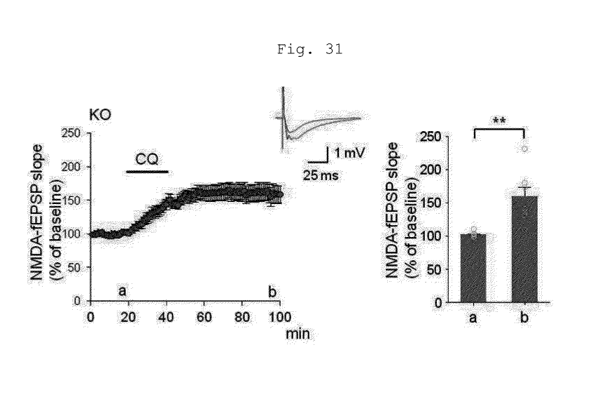

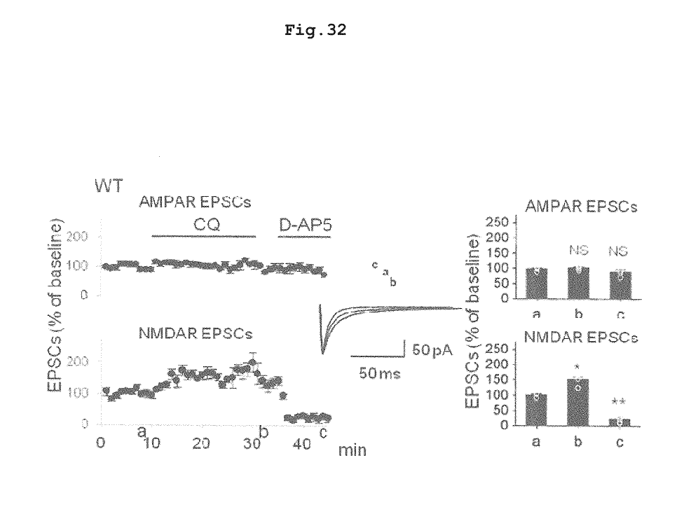

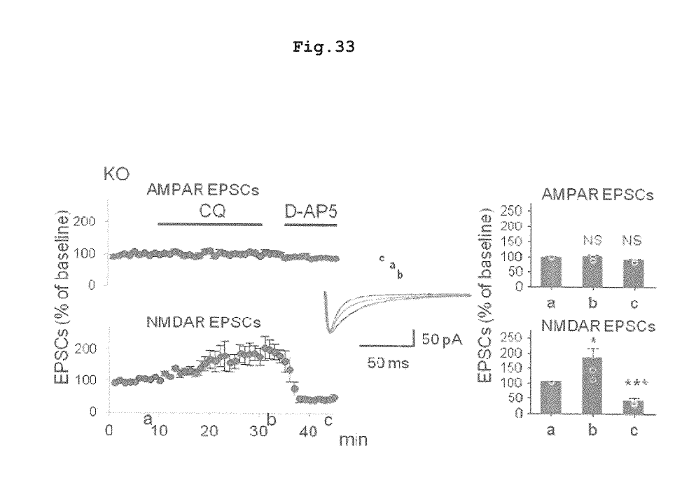

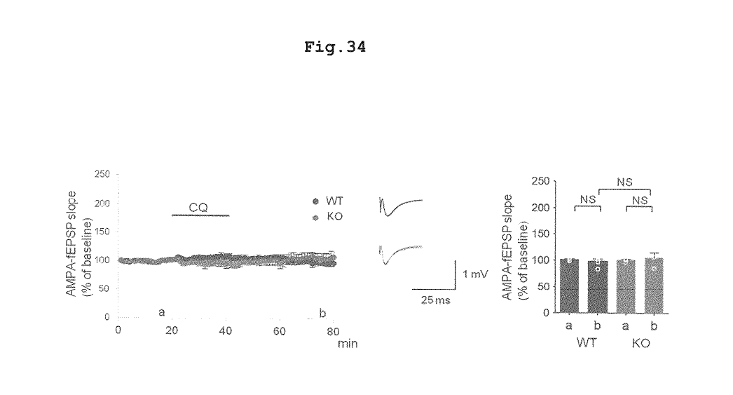

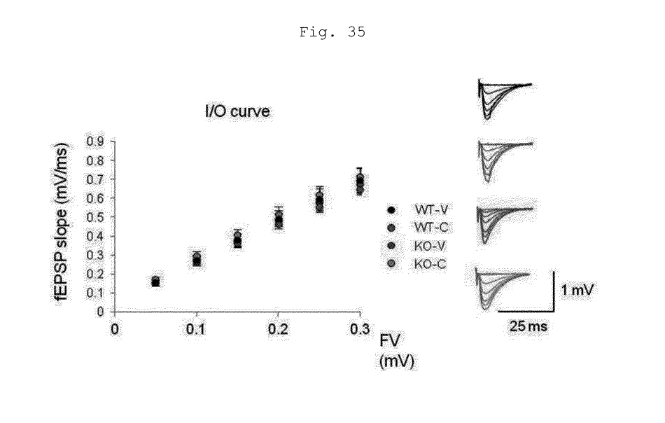

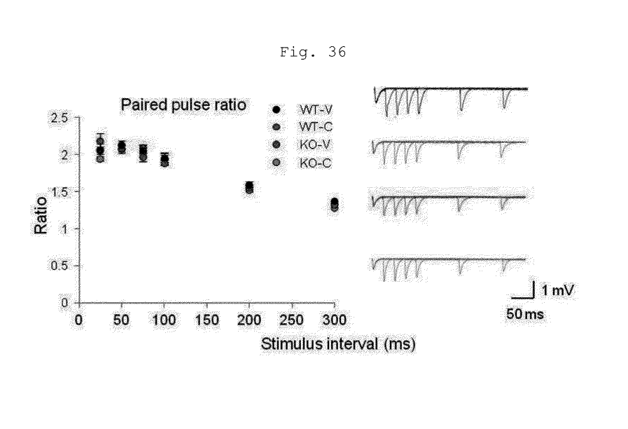

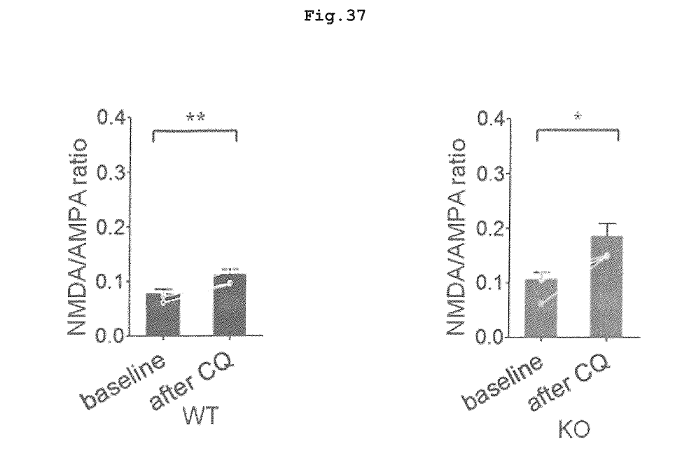

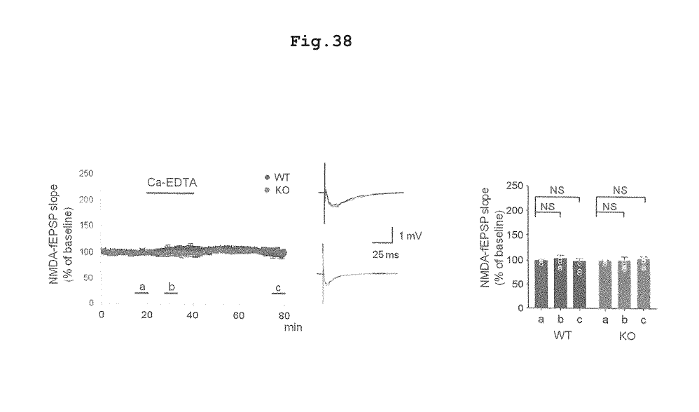

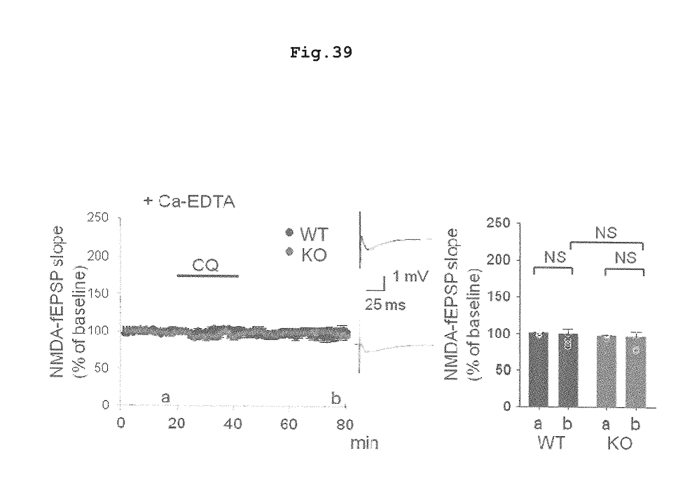

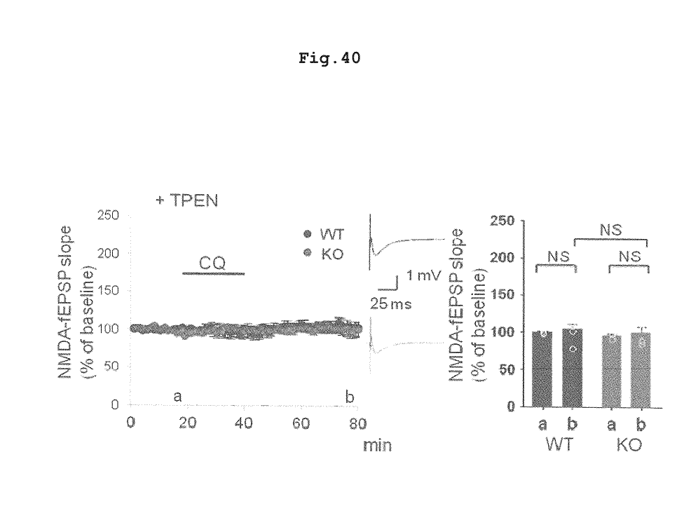

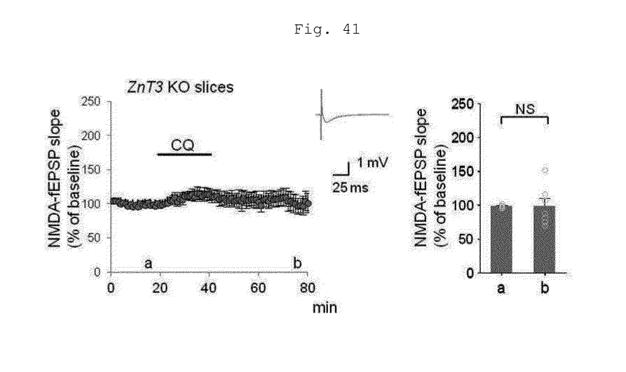

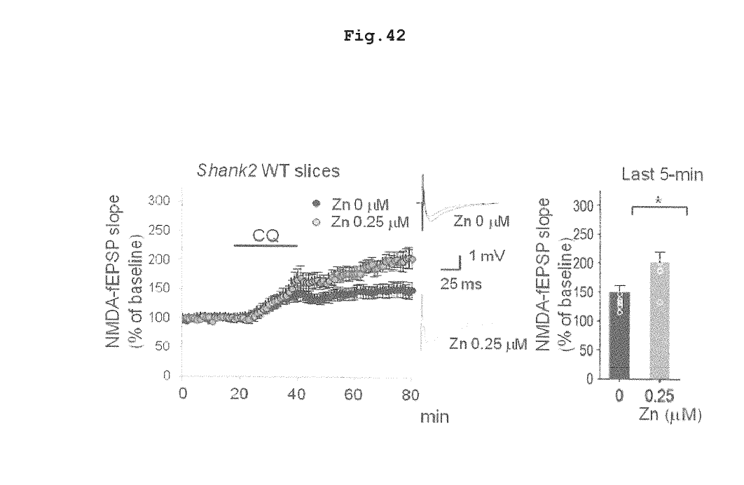

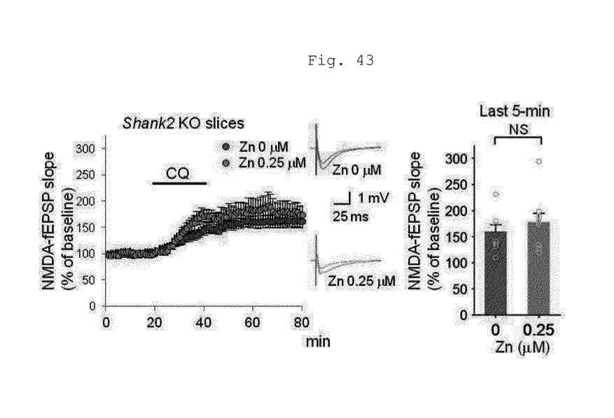

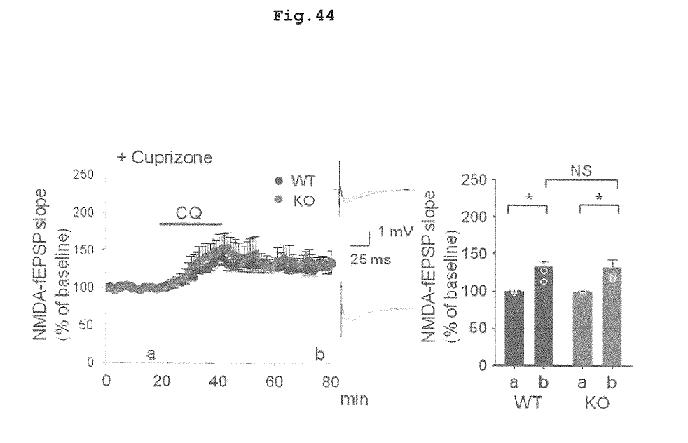

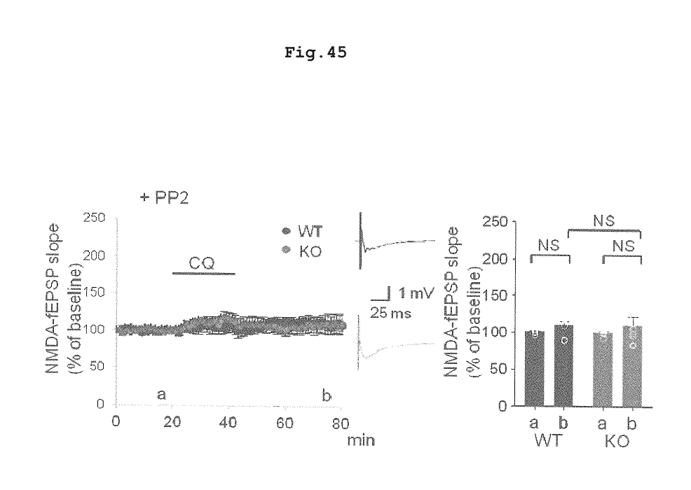

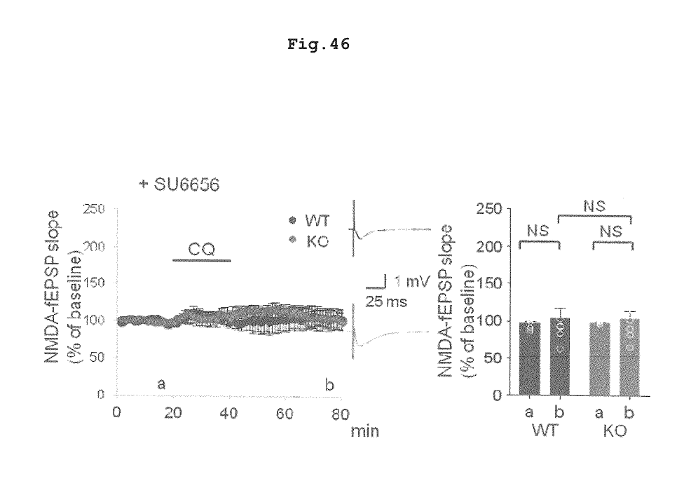

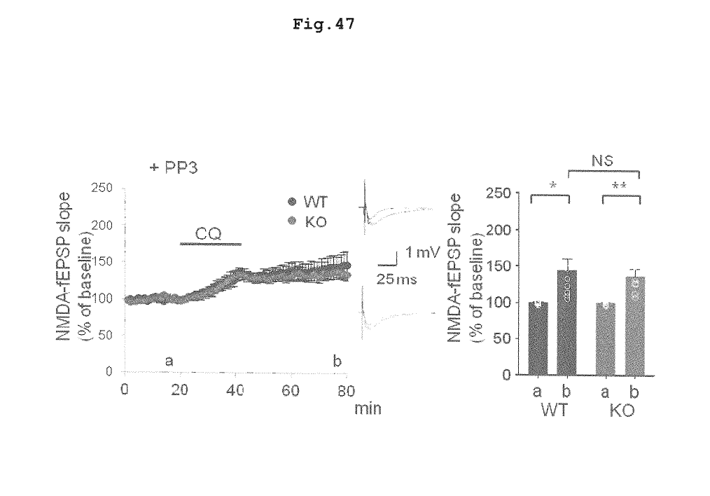

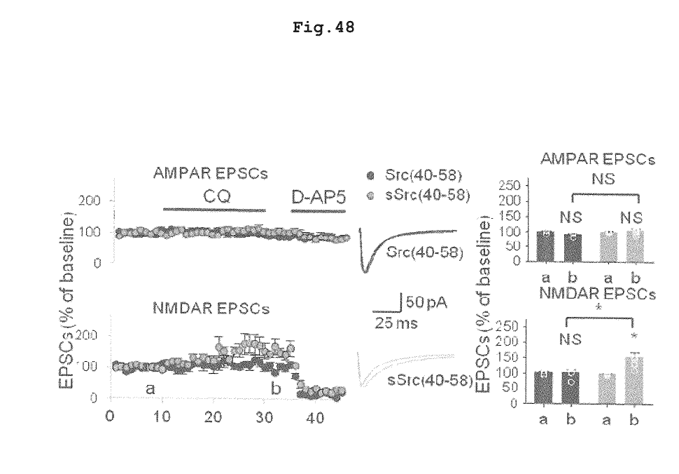

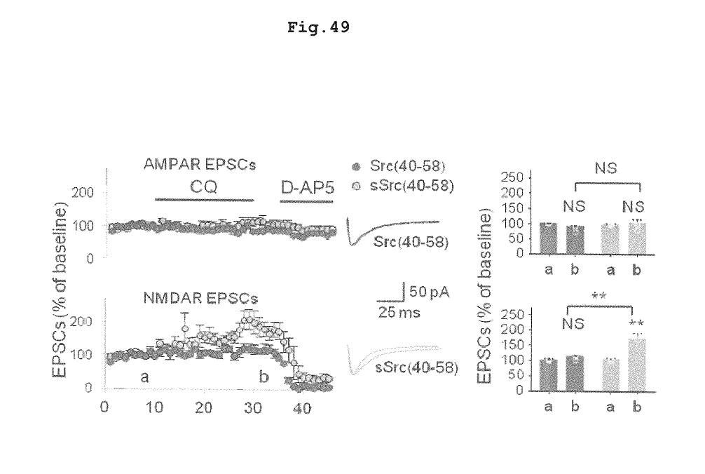

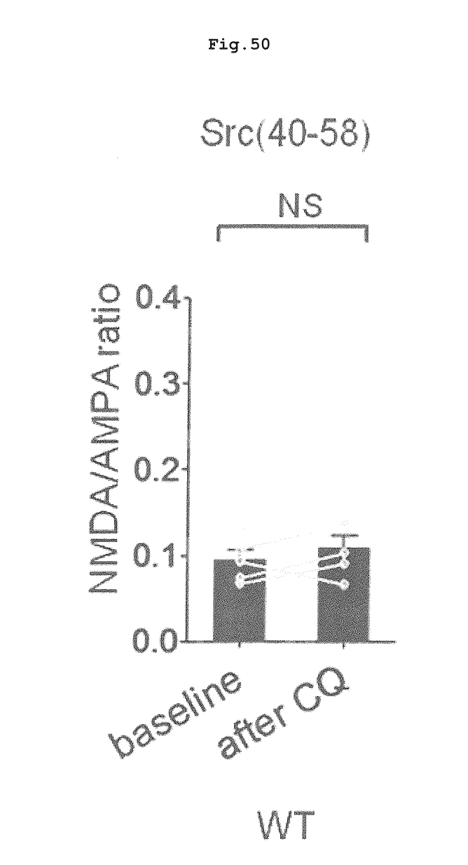

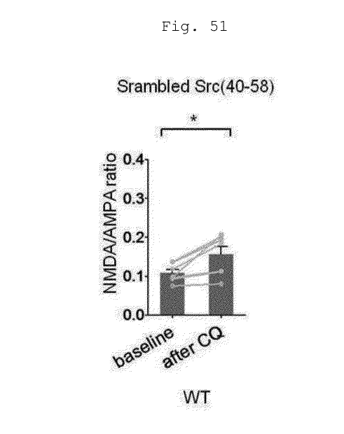

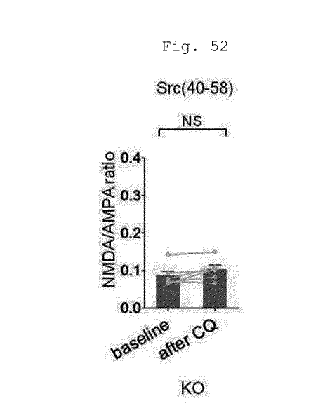

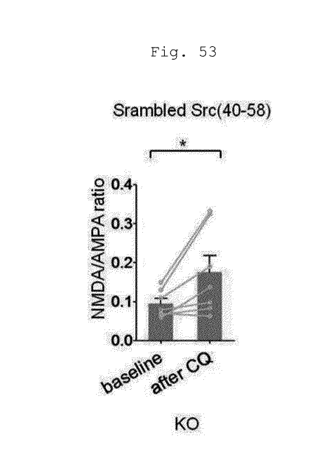

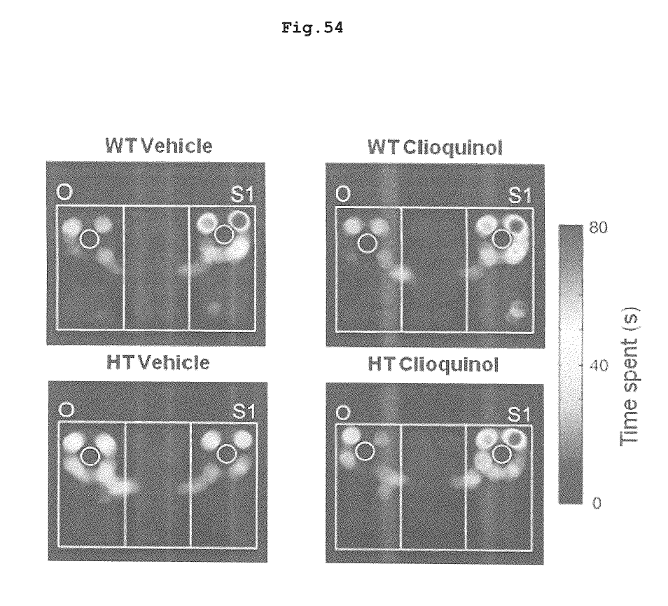

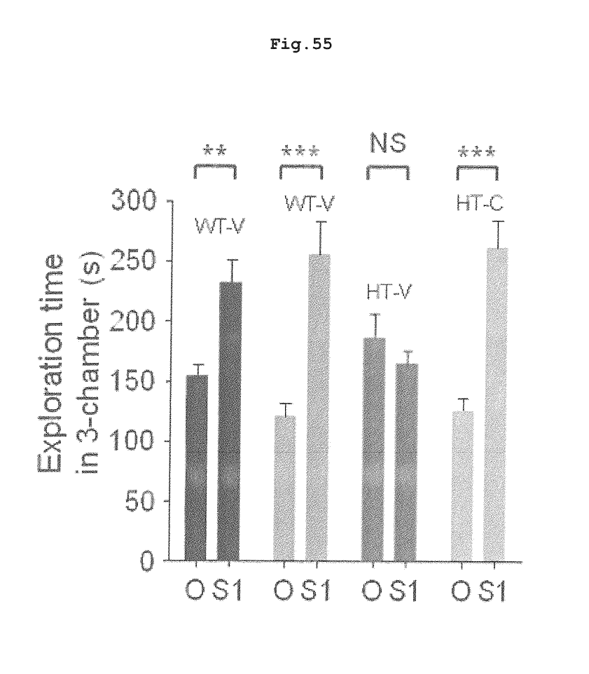

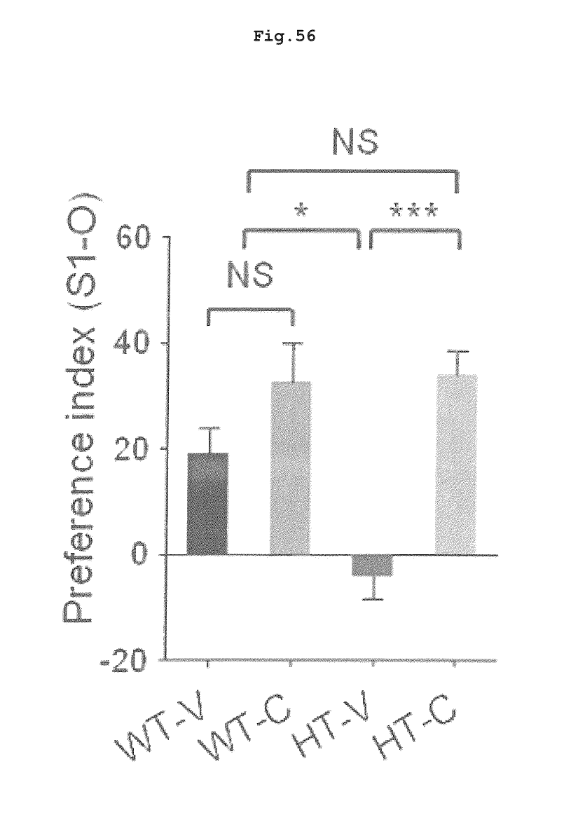

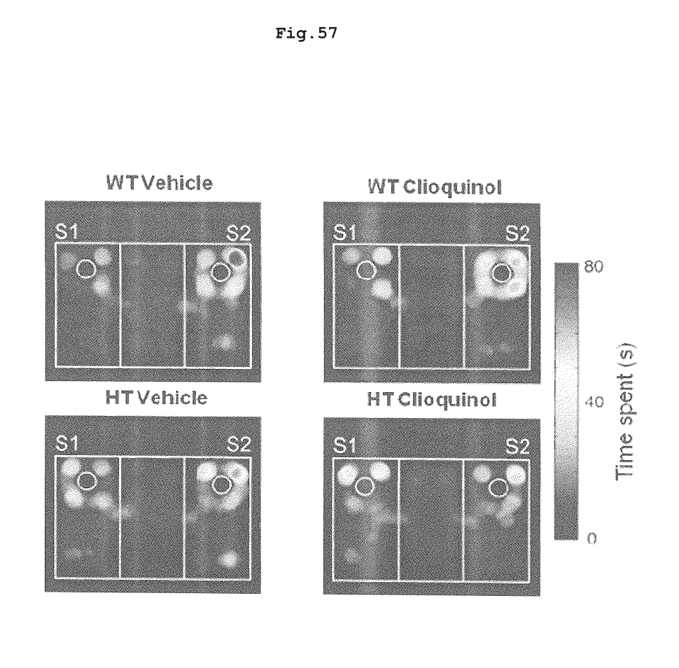

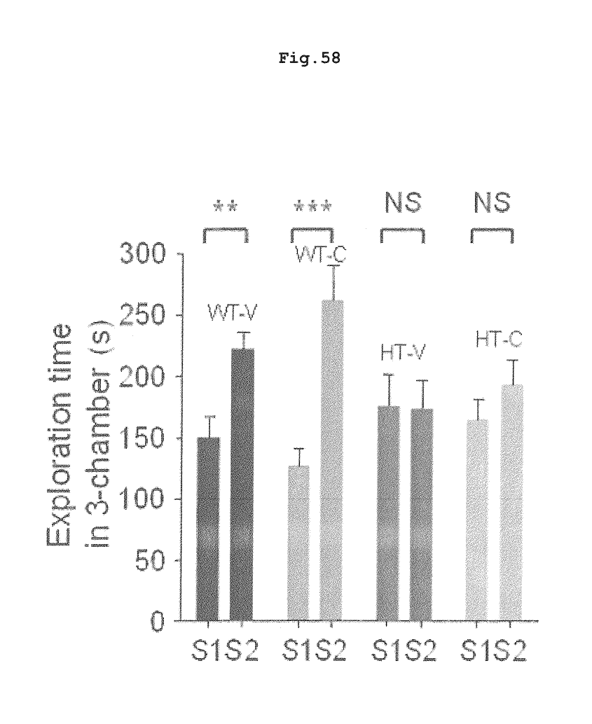

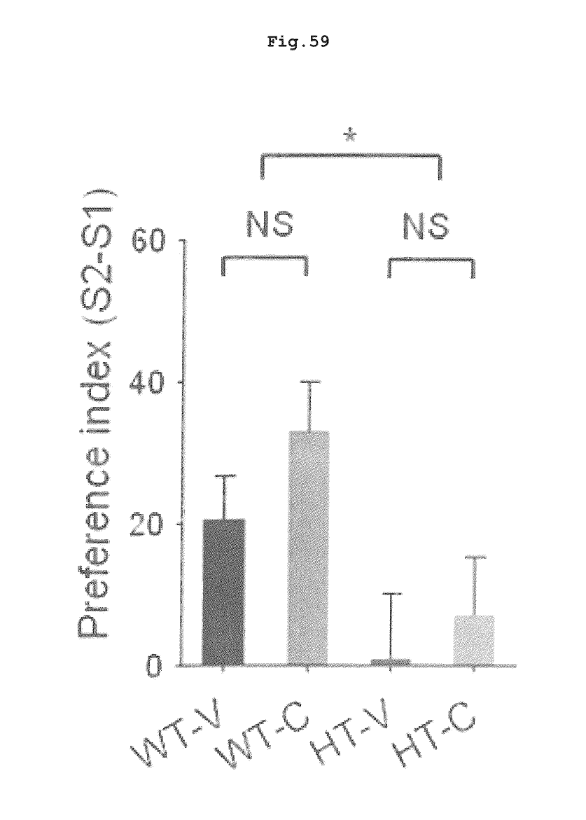

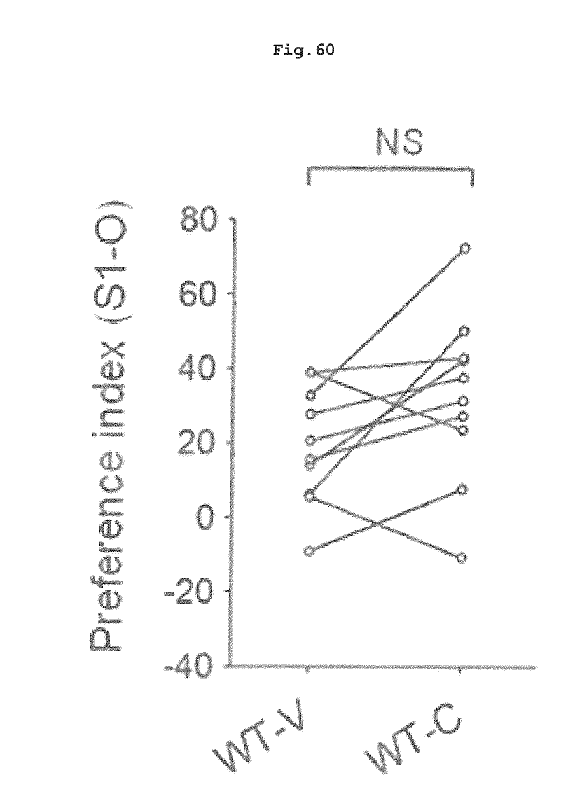

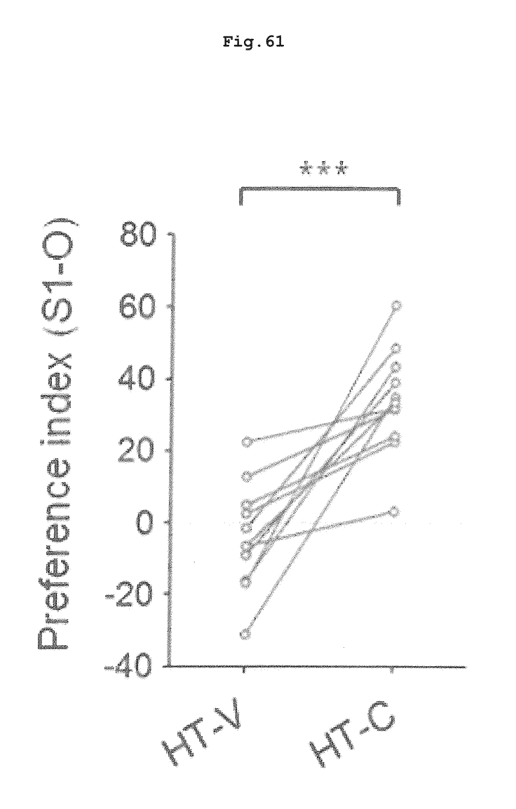

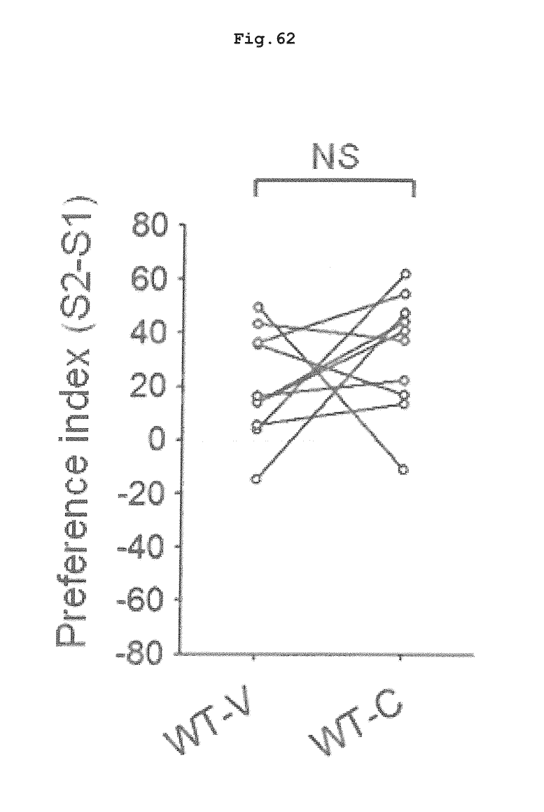

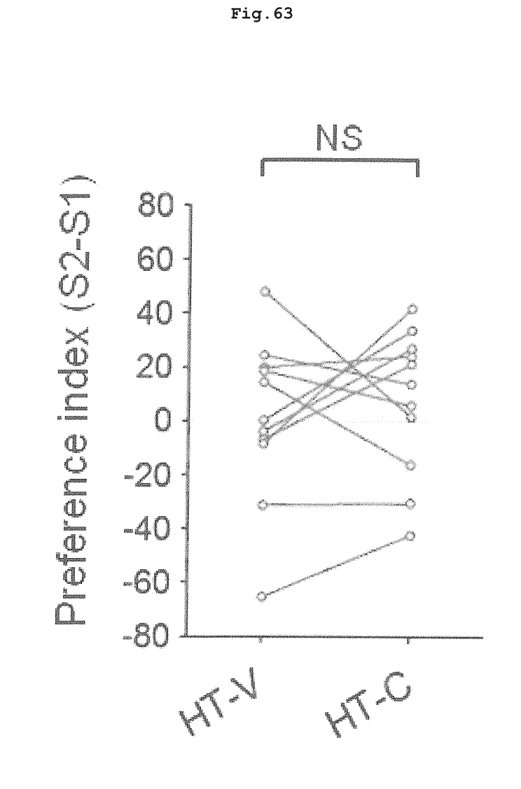

FIGS. 6 and 7

Clioquinol (CQ) did not improve excessive action in the shank2^{-/-} mice. (n=23 for WT-V and WT-C, 21 for KO-V and KO-C, *p<0.001, two-way ANOVA and Kruskal-Wallis one-way ANOVA with Dunn's post hoc test). Data are presented as mean±SEM.

FIGS. 8 to 19 show that clioquinol (CQ) improves social interactions in Shank2^{-/-} mice.

FIG. 8

A typical example of administrating a drug is shown. Animals were divided into two experimental groups. One group received the vehicle first and then the CQ, and the other group received the CQ first, and then the vehicle. The mice were tested 2 hours after the first injection (vehicle or 30 mg/kg of CQ; i.p.). After resting for 6 days in a single case, the mice were mutually treated and retested.

FIGS. 9 to 19

Clioquinol CQ improves social interaction (FIGS. 9 to 15) but has no effect on social novelty recognition (FIGS. 16 to 19) in Shank2^{-/-} (KO) mice, or on both social interaction and social novelty recognition in wild-type WT mice, as determined by the time spent exploring/sniffing targets (S1/stranger vs. O/object, or S2/new stranger vs. S1/previous stranger). Heat maps in (FIG. 16) represent examples of mouse movements. Data were analyzed as paired comparisons of the effects of CQ (before and after) within wild-type WT and knockout KO groups, or within the vehicle-first and CQ-first groups to minimize carryover effects. (n=9 for WT-V and WT-C (vehicle-first), n=8 for KO-V and KO-C (vehicle-first), n=9 for WT-V and WT-C (CQ-first), n=8 for KO-V and KO-C (CQ-first), NS indicates “not significant”, *p<0.05, **p<0.01, ***p<0.001, Student's t-test).

In FIGS. 20 to 22, free Zn Levels are similar in wild-type WT and Shank2^{-/-} brains, and CQ has no effect on ZnT3 protein levels.

FIG. 20

There was no difference in the total level of free Zn between wild-type WT and Shank2^{-/-} brains (8 weeks), as determined using the free Zn-binding fluorescent dye, TFL-Zn (N=15 hippocampal slices from three animals for WT and knockout (KO), NS indicates “not significant”, Student's t-test, ROI is region of interest).

FIG. 21

CQ treatment has no effect on the total level of ZnT3 protein in whole brain crude synaptosomal fractions from WT and Shank2^{-/-} mice (8 weeks). Mice were acutely injected with CQ (30 mg/kg; i.p.) 2 hours before sample preparation and immunoblot analysis. (n=4 for each group, NS indicates “not significant”, one-way ANOVA).

FIG. 22

Free Zn was undetectable in ZnT3^{-/-} brain (postnatal day 23), as determined by TFL-Zn.

FIGS. 23 to 27 show that clioquinol (CQ) does not improve repetitive actions and anxiety-like states in Shank2^{-/-} mice.

FIGS. 23 to 26

CQ (30 mg/kg; i.p.) injected 2 hours prior to testing fails to improve jumping and has no effect on grooming in Shank2^{-/-} mice (n=10 for WT-V and WT-C, n=11 for KO-V and for KO-C, NS indicates “not significant”, *p<0.05, Student's t-test).

FIG. 27

CQ has no effect on the time spent in the center region of the open field arena in WT and Shank2^{-/-} mice. (n=10 for WT-V and WT-C, 11 for KO-V and for KO-C, NS indicates “not significant”, *p<0.05, two-way ANOVA and Kruskal-Wallis one-way ANOVA with Dunn's post hoc test)

FIGS. 28 and 33 show that clioquinol CQ restores NMDAR (NMDA receptor) function in the Shank2^{-/-} synapses.

FIG. 28

Clioquinol (4 uM) normalizes the NMDA/AMPA ratio at Shank2^{-/-} hippocampal SC-CA1 synapses, as measured by NMDA- and AMPA-eEPSCs. Representative eEPSC traces were recorded at −70 mV and+40 mV. NMDA-eEPSCs were measured at +40 mV holding potential, 60 ms after the stimulation (n=14 cells (from 10 animals) for WT-V, n=11 cells (from 7 animals) for WT-C, n=12 cells (from 8 animals) for KO-V, and n=12 cells (from 7 animals) for KO-C, *p<0.05, One-way ANOVA with Tukey's post hoc test).

FIG. 29

As measured from fEPSP, CQ (4 uM) restored LTP included from tetanus (100 Hz) in Shank2^{-/-} hippocampal SC-CA1 synapses. (n=13 slices (from 8 animals) for WT-V, n=12 (from 5 animals) for WT-C, n=13 (from 7 animals) for KO-V, and n=11 (from 5 animals) for KO-C, NS is not significant, *p<0.05, One-way ANOVA with Tukey's post hoc test).

FIGS. 30 and 31

CQ (4 uM, 20 minutes) enhances NMDAR function at WT and Shank2^{-/-} hippocampal SC-CA1 synapses, as measured by NMDA-fEPSPs (n=8 slices (7 animals) for WT, and n=8 (7 animals) for KO, *p<0.01, Student's t-test). The labels a and b indicate 5-minute duration before CQ and the end of recording, respectively.

FIGS. 32 and 33

CQ (4 uM, 20 minutes) enhances NMDAR function at WT and Shank2^{-/-} hippocampal SC-CA1 synapses, as determined by simultaneous measurements of NMDA- and AMPA-eEPSCs at −40 mV. NMDA-eEPSCs were measured at 60 ms after stimulation. D-AP5 (50 uM, 10 minutes) was used to test NMDAR dependence. The labels a, b, and c indicate 5-minute duration before and after CQ, and at the end of recording, respectively (n=4 cells (3 slices) for WT, and 5 cells (4 slices) for KO, NS is not significant, *p<0.05, **p<0.01, ***p<0.001, Student's t-test, compared with the 5-minute duration before CQ). Data are presented as mean±SEM.

FIGS. 34 to 37 show that clioquinol CQ has no effect on AMPA-fEPSPs, input-output ratio, or paired pulse ratio, but increases the NMDA/AMPA ratio of eEPSCs at Shank2^{-/-} synapses.

FIG. 34

CQ (4 uM) has no effect on AMPA-fEPSPs. The labels a and b indicate 5-minute durations before CQ and the end of recordings, respectively (n=5 slices (4 animals) for WT and 5 (4) for KO, NS is not significant, Student's t-test).

FIG. 35

CQ (4 uM) has no effect on the input-output relationship at WT or Shank2^{-/-} hippocampal SC-CA1 synapses, as determined by plotting the initial slopes of AMPA-fEPSPs against amplitudes of fiber volley. (n=9 slices (7 animals) for WT-V, n=9 (7 animals) for WT-C, n=8 (5 animals) for KO-V, and n=9 (6 animals) for KO-C, one-way ANOVA).

FIG. 36

CQ (4 uM) has no effect on the paired pulse ratio at both WT and Shank2^{-/-} hippocampal SC-CA1 synapses, as determined by plotting the ratio of first/second initial slopes of AMPA-fEPSPs against interstimulus intervals. (n=9 slices (7 animals) for WT-V, n=9 (7 animals) for WT-C, n=8 (5 animals) for KO-V, and n=9 (6 animals) for KO-C, one-way ANOVA).

FIG. 37

CQ (4 uM) increases the NMDA/AMPA ratio of eEPSCs at −40 mV in both WT and Shank2^{-/-} hippocampal SC-CA1 synapses. (n=4 cells (3 animals) for WT, and n=5 (4 animals) for KO, *p<0.05, Student's t-test).

FIG. 38 shows that Ca-EDTA has no effect on NMDAR. Ca-EDTA (2 mM) has no effect on NMDAR function during and after the treatment, as measured by NMDA-fEPSPs. The labels a, b, and c indicate 5-minute duration before and during Ca-EDTA, and at the end of recording, respectively. (n=8 slices (5 animals) for WT, and n=10 (5 animals) for KO, NS is not significant, Student's t-test)

FIGS. 39 to 44 show than clioquinol (CQ)-dependent NMDAR activation requires Zn mobilization from pre- to postsynaptic sites.

FIGS. 39 and 40

CQ (4 uM, 20 minutes) fails to enhance NMDAR function at hippocampal SC-CA1 synapses in the presence of Ca-EDTA or TPEN (Zn chelators more potent than CQ), as measured by NMDA-fEPSPs. Shank2^{-/-} hippocampal slices were bath incubated with Ca-EDTA (2 mM) or TPEN (25 uM) throughout recordings. The labels a and b indicate 5-minute durations before CQ and the end of recordings, respectively (Ca-EDTA, n=6 slices (3 animals) for WT, n=6 (3 animals) for KO, NS is not significant, Student's t-test, TPEN, n=6 slices (2 animals) for KO and WT, NS is not significant, Student's t-test).

FIG. 41

CQ fails to enhance NMDAR function at ZnT3^{-/-} hippocampal SC-CA1 synapses (n=7 slices (3 animals), NS is not significant, Student's t-test).

FIGS. 42 and 43

Exogenously added Zn (0.25 uM) enhances NMDAR function at WT but not Shank2^{-/-} synapses. Additional Zn was bath applied throughout the recording. (n=8 slices (7 animals) for 0 uM, n=7 slices (4 animals) for 0.25 uM in WT, and n=8 (5 animals) for 0 uM, n=9 (4 animals) for 0.25 uM in KO, NS is not significant, *p<0.05, Student's t-test).

FIG. 44

CQ enhances NMDAR function in the presence of cuprizone, a Cu²⁺-specific chelator. (n=5 slices (4 animals) for WT and n=5 (4 animals) for KO, NS is not significant, *p<0.05, Student's t-test). Data are presented as mean±SEM.

FIGS. 45 to 49 show that trans-synaptic Zn mobilization activates NMDARs through Src family kinases.

FIGS. 45 to 47

CQ (4 uM, 20 minutes) fails to enhance NMDAR function at Shank2^{-/-} SC-CA1 synapses in the presence of PP2 or SU6656, but effectively enhances NMDAR function in the presence of PP3, an inactive PP2 analogue, as measured by NMDA-fEPSPs. Hippocampal slices were bath incubated with PP2 (10 uM) or SU6656 (10 uM) throughout recordings (PP2, n=7 slices (6 animals) for WT and n=7 (5 animals) for KO; SU6656, n=7 (5 animals) for WT and n=7 (4 animals) for KO; PP3, n=8 (4 animals) for WT and n=8 (5 animals) for KO, NS is not significant, *p<0.05, **p<0.01, Student's t-test).

FIGS. 48 and 49

CQ fails to enhance NMDAR function at Shank2^{-/-} SC-CA1 synapses (FIG. 49) in the presence of Src (40-58), a specific peptide inhibitor of Src, but effectively enhances NMDAR function in the presence of sSrc (40-58), a scrambled version of the peptide, as determined by simultaneous measurements of NMDA- and AMPA-eEPSCs at −40 mV. NMDA-eEPSCs were measured at 60 ms after the stimulation. FIG. 49 shows WT mice, and FIG. 49 shows Shank2^{-/-} mice. The labels a and b indicate 5-minute durations before CQ and the end of recordings, respectively (Src (40-58), n=5 cells (4 animals) for WT and n=6 (4 animals) for KO; sSrc (40-58), n=7 (5 animals) for WT, n=7 (6 animals) for KO, NS is not significant, *p<0.05, **p<0.01, Student's t-test, compared with the 5-minute duration before CQ). Data are presented as mean±SEM.

FIGS. 50 to 53 show that Src inhibitory peptide blocks CQ-dependent NMDAR activation.

Src-inhibitory peptide, Src (40-58), but not its scrambled version, sSrc (40-58), blocks CQ-dependent NMDAR activation, as measured by the NMDA/AMPA ratio at −40 mV. (Src (40-58), n=5 cells (4 animals) for WT and n=6 (4 animals) for KO; sSrc (40-58), n=7 (5 animals) for WT and n=7 (6 animals) for KO, NS is not significant, *p<0.05, Student's t-test).

FIGS. 54 to 59 show that trans-synaptic Zn mobilization improves social interactions in Tbr1^{+/-} mice. CQ enhances social interaction (FIGS. 54 to 56) in Tbr1^{+/-} mice, but fails to enhance social novelty recognition (FIGS. 57 to 59), and has no effect on WT mice. This was measured by time spent in exploring targets and the social preference index calculated from the result. The heat map of FIGS. 54 and 57 show an example of the movement of mice. (n=10 for WT-V and WT-C, n=11 for HT-V and HT-C, NS is not significant, *p<0.05, **p<0.01, ***p<0.001, two-way ANOVA and one-way ANOVA with Tukey's post hoc test). Data are presented as mean±SEM.

FIGS. 60 to 63 show that clioquinol (CQ) improves social interactions in Tbr^{+/-} (HT) mice. CQ improves social interaction (FIG. 61) but has no effect on social novelty recognition (FIG. 63) in Tbr1^{+/-} mice, and on both parameters (social interaction and social novelty recognition) in WT mice (FIGS. 60 and 62), as determined by the times spent in exploring the targets (S1/strangers vs. O/object, or S2/new stranger vs. S1/old stranger). The paired comparisons of the effects of CQ (before and after) within the WT or HT group were made to minimize carryover effects (n=10 for WT-V and WT-C, n=11 for HT-V and HT-C, NS is not significant, ***p<0.001, Student's t-test).

### DETAILED DESCRIPTION OF THE INVENTION

The present invention provides a pharmaceutical composition for treating autism spectrum disorders, the pharmaceutical composition including clioquinol or pharmaceutically acceptable salt thereof.

In addition, the present invention relates to a food composition for improving autism spectrum disorders, the food composition including clioquinol or pharmaceutically acceptable salt thereof.

Further, the present invention relates to a method of treating and improving autism spectrum disorders, the method including administering clioquinol or pharmaceutically acceptable salt thereof.

In addition, the present invention relates to a treatment and an improvement of autism spectrum disorders through clioquinol or pharmaceutically acceptable salt thereof.

Hereinafter, the present invention will be described in detail.

Clioquinol

The present invention relates to a pharmaceutical composition and/or a food composition including clioquinol or pharmaceutically acceptable salt thereof. In the present invention, clioquinol treats/improves autism spectrum disorder by enhancing postsynaptic NMDA receptor functions. Clioquinol of the present invention preferably enhances NMDA receptor functions through Zn ionophore activation, and clioquinol enhances the NMDA receptor functions through Src family tyrosine kinase. In addition, clioquinol promotes Zn mobilization in the synapses, and clioquinol preferably allows Zn to transfer from pre-synaptic Zn pools to a postsynaptic sites, so that the NMDAR function is enhanced through Src family tyrosine kinase (SFK). Thus, the NMDAR function is normalized.

By administrating clioquinol or pharmaceutically acceptable salt thereof of the present invention to the patient, the social interaction deficiency of the patient is improved.

Autism Spectrum Disorder

The autism spectrum disorder of the present invention signifies autism spectrum disorder generally known in the medical field. This involves social interaction disorders, communication disorders, limited and repetitive actions, neural development disorders, etc.

Composition Including Clioquinol

The present invention relates a pharmaceutical composition or a food composition including clioquinol or pharmaceutically acceptable salt thereof. Clioquinol and the pharmaceutically acceptable salt thereof may be obtained by methods generally known in the arts without specific limitations.

Pharmaceutical Composition

The pharmaceutical composition of the present invention may include 0.01 to 80 weight % of clioquinol or pharmaceutically acceptable salt thereof, and may preferably include 0.02 to 65 weight %. However, this may be increased or decreased according to the needs of the person to be administrated, and may be adequately increased or decreased according to conditions of dietary life, nutrition state, progression of the disease, amount of inflammation, etc.

The pharmaceutical composition of the present invention maybe administered orally or parenterally, and may be generally used in a form of a pharmaceutical medicine. A pharmaceutical preparation preferably includes an oral administration preparation such as a tablet, a soft or hard capsule, liquid, a suspension, etc. and the pharmaceutical preparation may be prepared by using the pharmaceutically allowable carrier, for example, in the case of the oral administration preparation, excipents, binders, disintegrants, glydents, solubilizers, suspending agent, preserved agents, extenders, etc. In addition, when the pharmaceutical composition of the present invention is administered parenterally, subcutaneous infusion through injection or transdermal absorption through patches are preferable, but not limited thereto.

The administration amount of the pharmaceutical composition including clioquinol or pharmaceutically acceptable salt thereof of the present invention may be determined by an expert according to various factors such as the state, age, sex, complications, etc. of the patient, and generally, 0.1 mg to 10 g per 1 kg, and more preferably 10 mg to 5 g may be administered for an adult. The amount may be appropriately increased or decreased according to weight, age, normal physical state, sex, diet, administration route, etc. of the patient such minorities, children or infants. In addition, 1 day amount or ½, ⅓ or ¼ amount of the 1 day amount of the pharmaceutical composition maybe included in a dosage unit, and the pharmaceutical composition may be administered 1 to 6 times a day.

Food Composition

The present invention relates to a food composition for treating autism spectrum disorders, the food composition including clioquinol or acceptable salt thereof.

The food of the present invention may include dietary supplements, health functional foods, functional foods, etc., but is not limited thereto, and also include adding the composition of the present invention or acceptable salt thereof to natural foods, processed foods, normal food materials, etc.

In the food composition of the present invention, clioquinol or acceptable salt thereof may be added as itself or may be used with other food or food compositions, and may be appropriately used according to typical methods. The mixed amount of the active component may be appropriately determined according to the purpose of use (prevention, improvement or therapeutic methods). In general, 0.1 to 70 weight % of clioquinol or pharmaceutically acceptable salt thereof, and may preferably include 0.2 to 50 weight % maybe added to 100 weight % of ingredients of food or drink when manufacturing the food or the drink. The active amount of the compound or acceptable salt thereof of the present invention may be used based upon the active amount of the pharmaceutical composition, but may be below the range in the case of long-term intakes for the purpose of health and hygiene or the purpose of adjusting health. The acceptable salt signifies pharmaceutically acceptable salt.

There is no specific limitation for the types of the food. The food composition may be used as a form of an oral administration preparation such as a tablet, a soft or hard capsule, liquid, a suspension, etc. and the preparation may be prepared by using the pharmaceutically allowable carrier, for example, in the case of the oral administration preparation, excipents, binders, disintegrants, glydents, solubilizers, suspending agent, preserved agents, extenders, etc.

Patient

The patient administered with clioquinol or pharmaceutically acceptable salt thereof of the present invention signifies the patient diagnosed with autism spectrum disorder or suspected of having autism spectrum disorder. The patient may be an adult or a minor, and may be a human or a mammal .

Advantages and features of the present invention, and method for achieving thereof will be apparent with reference to the examples that follow. But, it should be understood that the present invention is not limited to the following embodiments and may be embodied in different ways, and that the embodiments are given to provide complete disclosure of the invention and to provide thorough understanding of the invention to those skilled in the art, and the scope of the invention is limited only by the accompanying claims and equivalents thereof.

<Material and Method>

Mice

Shank2^{-/-} mice and Tbr1^{+/-} mice are reported to be autism model animals (Won H, et al. Autistic-like social behavior in Shank2-mutant mice improved by restoring NMDA receptor function. Nature 486, 261-265 (2012), Huang T N, et al. Tbr1 haploinsufficiency impairs amygdalar axonal projections and results in cognitive abnormality. Nat Neurosci 17, 240-247 (2014)). All mice were backcrossed into a C57BL/6 background, and housed and bred in a mouse vivarium at the Korea Advanced Institute of Science and Technology (KAIST, Shank2^{-/-}mice) and at Academia Sinica (Tbr1^{+/-} mice). Pups were kept with the dam until weaning at postnatal day 21. After weaning, animals were group housed in mixed-genotype groups of 3 to 5 mice per cages, and randomly subjected to electrophysiological and behavioral experiments. Animals at 3 to 5 weeks of age were used for electrophysiological experiments; male animals at 2 to 4 months of age were used for behavioral assays. For TFL-Zn staining, male animals at 8 weeks (for Shank2^{-/-}mice) were used. Wild-type littermates were used as controls.

ZnT3^{-/-} mice have been reported (Lee J-Y, Cole T B, Palmiter R D, Suh S W, Koh J-Y. Contribution by synaptic zinc to the gender-disparate plaque formation in human Swedish mutant APP transgenic mice. Proceedings of the National Academy of Sciences 99, 7705-7710 (2002)), and was maintained in the facility in KAIST. These mice also were backcrossed into the C57BL/6 background. Both male and female animals at 3 to 5 weeks were used for electrophysiological experiments, and TFL-Zn staining (P23).

All mice were bred and maintained according to the KAIST and Academia Sinica Animal Research Requirements, and all procedures were approved by the Committees of Animal Research at KAIST, and at Academia Sinica. Mice were fed ad libitum by standard rodent chow and tap water, and housed under 12-hour light/dark cycle (lights off at 19:00 in KAIST, at 20:00 in Academia Sinica).

Clioquinol

Clioquinol (Calbiochem) was dissolved in DMSO (Sigma) and polyethylene glycol (Aldrich) (DMSO:PEG=1:9) to a final concentration of 20 g/L. Wild-type and Shank2^{-/-} mice (or Tbr1^{+/-} mice) received acute intraperitoneal injection of clioquinol (30 mg/kg), or the same volume of DMSO-PEG mixture. The injection was performed 2 hours before (Shank2^{-/-} mice and wild-type littermates), or 3 hours before (Tbr1^{+/-} mice and wild-type littermates) behavioral assays at the discretion of the facility.

Drug Treatment Scheme

A within-subjects design was designed with a one week washout period (FIG. 8), and divided animals into two groups, vehicle-first and clioquinol-first group, to rule out carryover effects. Each mouse received a single acute dose of vehicle or clioquinol, and underwent a single behavioral task, one task per week. Testing was conducted in dedicated behavioral test rooms during the light phase (three-chamber test) and the dark phase (repetitive behaviors and open field test).

Three-Chamber Social Interaction Assay

The three-chamber social interaction assay for Shank2 mice (Shank2^{-/-}and wild-type littermates) and Tbr1 mice (Tbr1^{+/-} and wild-type littermates) were performed (Won H, et al. Autistic-like social behavior in Shank2-mutant mice improved by restoring NMDA receptor function. Nature 486, 261-265 (2012), Huang T N, et al. Tbr1 haploinsufficiency impairs amygdalar axonal projections and results in cognitive abnormality. Nat Neurosci 17, 240-247 (2014)). In short, the assay consisted of three phases of 10 minutes duration: habituation, social interaction (Stranger1 vs. Object), and social novelty recognition (Stranger 1 vs. Stranger 2). Exploration was defined as each instance in which wild-type or mutant mouse tries to sniff Object/Stranger, or orients its nose towards and come close to object/stranger. Individual movement tracks were analyzed by Ethovision 10.0 (Noldus) and modified by custom-designed software MatLab (Mathworks) to generate heat maps. Time spent in exploration was analyzed by the researcher who was blinded to the subject genotype (in Shank2 mice), or by using the Smart Video Tracking System (Panlab, in Tbr1 mice). In addition to exploration time, the preference index, which represents a numerical difference between time spent exploring the targets (Stranger1 vs. Object, or Stranger 2 vs. Stranger 1) divided by total time spent exploring both targets, as described previously. (Won H, et al. Autistic-like social behavior in Shank2-mutant mice improved by restoring NMDA receptor function. Nature 486, 261-265 (2012)).

Repetitive Behaviors

Shank2^{-/-} mice and their wild-type littermates in their home cages without bedding were used to measure times spent in repetitive behaviors including jumping and grooming. In this case, jumping is defined as mice planting rear feet and standing up by lifting front feet along the cage, in other words, the cage corner or a side wall, and jumping up such that both of the rear feed are separated from the bottom surface. Meanwhile, grooming is defined as stroking or scratching the face or the body with two front feet or licking a part of the body. The experiments and analysis were performed independently in a blind manner.

Open Field Test

The size of the open field box was 40×40×40 cm, and the center zone line was 13.3 cm apart from the edge. Mice were placed in the center in the beginning of the test, and mouse movements were recorded with a video camera for 60 minutes, and analyzed by Ethovision 10.0 (Noldus).

Electrophysiology

For electrophysiological experiments, sagittal hippocampal slices (400 uM thick for extracellular, 300 uM thick for intracellular recordings) of Shank2^{-/-} mice (or ZnT3^{-/-} mice) and their wild-type littermates were prepared using a vibratome (Leica VT1200) in ice-cold dissection buffer containing (in mM) 212 sucrose, 25 NaHCO3, 5 KCl, 1.25 NaH2PO4, 0.5 CaCl2, 3.5 MgSO4, 10 D-glucose, 1.25 L-ascorbic acid, and 2 Na-pyruvate bubbled with 95% 02/5% CO2. CA3 was removed to prevent epileptiform activity. Slices were restored for 1 hour in 32° C. in a normal artificial CSF (ACSF) (In units of mM: 124 NaCl, 2.5 kCl, 1 NaH2PO4, 25 NaHCO3, 10 glucose, 2 CaC12, 2 Mg504 oxidized by 95% 02/5% CO2) For the recording, a single slice was moved to and maintained in submerged-type chamber at 28° C., continuously perfused with ACSF (2 mL/minute) saturated with 95% 02/5% CO2. Stimulation and recording pipettes were pulled from borosilicate glass capillaries (Harvard Apparatus) using a micropipette electrode puller (Narishege).

For extracellular recordings, mouse hippocampal slices at the age of postnatal day 21 to 35 were used. Field excitatory postsynaptic potentials (fEPSPs) were recorded in the stratum radiatum of the hippocampal CA1 region using pipettes filled with ACSF (1 MΩ). fEPSP was amplified (Multiclamp 700B, Molecular Devices), and digitized (Digidata 1440A, Molecular

Devices) for measurements. The Schaffer collateral pathway was stimulated every 20 seconds with pipettes filled with ACSF (0.3 to 0.5 MΩ). The stimulation intensity was adjusted to yield a half-maximal response, and three successive responses were averaged and expressed relative to the normalized baseline. To induce LTP, high frequency stimulation (100 Hz, 1 s) was applied after a stable baseline was acquired. Clioquinol (4 uM) was bath-applied prior to and after LTP induction during the whole experimental processes. To isolate NMDAR-mediated fEPSPs, ACSF containing 2 mM calcium, 0.1 mM magnesium, and 6,7-dinitroquinoxaline-2,3-dione (10 uM, DNQX, Tocris), which inhibits AMPAR-mediated EPSPs, were used.

Whole-cell patch-clamp recordings of hippocampal CA1 pyramidal neurons were also made using a MultiClamp 700B amplifier (Molecular Devices) and Digidata 1440A (Molecular Devices). During whole-cell patch-clamp recordings, series resistance was monitored each sweep by measuring the peak amplitude of the capacitance currents in response to short hyperpolarizing step pulse (5 mV, 40 ms); only cells with a change in <25% were included in the analysis. For NMDA (N-Methyl-D-aspartate)/AMPA (alpha-amino-3-hydroxy-5-methylisoxazole-4-propionic acid) ratio experiments, mouse hippocampal slices at the age of postnatal day 17 to 25 were used. The recording pipettes (2.5 to 3.5 MΩ) were filled with an internal solution containing the following (in mM): 100 CsMeSO4, 10 TEA-Cl, 8 NaCl, 10 HEPES, 5 QX-314-C1, 2 Mg-ATP, 0.3 Na-GTP, 10 EGTA, with pH 7.25, 295 mOsm). CA1 pyramidal neurons were voltage-clamped at −70 mV, and EPSPs were evoked at every 15 sec. AMPAR-mediatedEPSCs were recorded at −70 mV, and 30 consecutive responses were recorded after stable baseline. After recording AMPAR-mediated EPSPs, holding potential was changed to +40 mV to record NMDA receptor-mediated EPSPs. NMDA component was measured at 60 ms after the stimulation. The NMDA/AMPA ratio was determined by dividing the mean value of 30 NMDA components of EPSPs by the mean value of 30 AMPAR-mediated EPSC peak amplitudes. Clioquinol (4 uM) was bath-applied from 20 minutes prior to and during the whole period of NMDA/AMPA ratio recording. For measuring AMPAR-mediated and NMDAR-mediated EPSCs together upon CQ treatment, pyramidal neurons were voltage-clamped at −40 mV, and EPSCs were evoked at every 15 sec. AMPAR-mediated EPSC was determined as a peak amplitude of EPSC, and NMDAR-mediated EPSC as a component at 60 ms after stimulation. NMDA/AMPA ratio at −40 mV was calculated by using both values, and monitored during the experimental process. Src-inhibiting peptide, Src (40-58), and its analogue, scrambled Src (40-58) (Lu Y M, Roder J C, Davidow J, Salter M W. Src activation in the induction of long-term potentiation in CA1 hippocampal neurons. Science (New York, N.Y.) 279, 1363-1367 (1998)) were purchased from Peptron, and introduced into the internal solution at a concentration of 0.03 mg/mL to observe whether Src-inhibition affects the action of Clioquinol. Picrotoxin (100 uM) was always added to ACSF to block GABAA receptor-mediated currents.

Data were acquired by Clampex 10.2 (Molecular Devices) and analyzed by Clampfit 10 (Molecular Devices). Drugs were purchased from Tocris (DNQX, TPEN, PP2, PP3, and D-AP5), and Sigma (picrotoxin, Ca-EDTA, cuprizone, and SU6656).

TFL-Zn staining

Brain sections (10 uM thick) were stained, without fixation, with the zinc-specific dye TFL-Zn (N-(6-Methoxy-8-quinolyl)-p-carboxybenzoylsulfonamide; 0.1 mM, Calbiochem) dissolved in phosphate buffered saline (pH 7.2), and photographed with a digital camera linked to a fluorescence microscope (Olympus IX71; excitation, 330 to 385 nm; dichromatic, 400 nm; barrier, 420 nm). Fluorescence signals were obtained using an image program (Image-Pro Insight, Media Cybernetics, Silver Spring, Md.). Images of five consecutive hippocampal slices from an individual brain were quantified using MetaMorph (Molecular Devices). Regions of interest (ROI) were defined as three to five squares (50×50 um²) in wild-type and Shank2^{-/-} hippocampal DG, CA3, and CA1 regions (5, 3, and 5 squares, respectively). For quantification, the total fluorescence from a Shank2^{-/-} ROI was normalized to that from an equivalent wild-type ROI.

Crude Synaptosome

Crude synaptosomes were prepared from Shank2^{-/-}mice (Won H, et al. Autistic-like social behavior in Shank2-mutant mice improved by restoring NMDA receptor function. Nature 486, 261-265 (2012)). In other words, mouse brains (2 months old) were homogenized in ice-cold homogenization buffer (0.32 M sucrose, 10 mM HEPES pH 7.4, 2 mM EDTA, protease inhibitors, phosphatase inhibitors). Homogenates were centrifuged at 900×g for 10 minutes. The resulting supernatant was centrifuged again at 12,000×g for 15 minutes. The pellet was resuspended in homogenization buffer and centrifuged at 13,000×g for 15 minutes (the resulting pellet is P2; crude synaptosomes).

Statistical Analysis

Statistical analysis is executed by using Prism GraphPad (Version 5.03) software.

### Experimental Example 1

### Evaluation of Social Interaction of Shank2^{-/-} Mice

Shank2^{-/-} mice were intraperitoneally injected by systemic administration of clioquinol (CQ, 30 mg/kg), an ionophore and a lipophilic Zn chelator (Kd≈10⁻⁷), which easily cross over blood brain barrier and mobilize Zn to a lower concentration slope. (Bareggi S R, Cornelli U. Clioquinol: review of its mechanisms of action and clinical uses in neurodegenerative disorders. CNS Neurosci Ther 18, 41-46 (2012)). Two hours after CQ treatment, the mice were subjected to a three-chamber social interaction test, which compares the preference of a mouse for a stranger mouse versus a novel inanimate object.

As a result, Shank2^{-/-} mice displayed reduced social interaction compared with wild-type (WT) mice, as determined by exploration/sniffing time and the derived social preference index (see figure legend for details) in vehicle-treated mice (FIGS. 1, 2, 8 to 15), consistent with previous results from Shank2^{-/-} and wild-type (WT) mice not treated by clioquinol . This impairment was improved by clioquinol treatment. In contrast, social interaction in wild-type mice was not affected by clioquinol.

When the social novelty recognition was determined in the 3-chamber test, by measuring the preference between the stranger mouse, which with the mouse before, and a new stranger mouse, Shank2^{-/-} mice displayed social novelty recognition corresponding to the wild-type mice (FIGS. 3, 16 to 19). In addition, clioquinol had no effect on social novelty recognition in both wild-type and Shank2^{-/-} mice.

The differential clioquinol-dependent improvement of social interaction in wild-type and Shank2^{-/-} mice is unlikely attributable to differences in the amount of free Zn available for clioquinol binding in these mice, because total levels of free Zn measured with the fluorescent dye TFL-Zn were not different (FIG. 20). In addition, the levels of zinc transporter (ZnT3) (Cole T B, Wenzel H J, Kafer K E, Schwartzkroin P A, Palmiter R D. Elimination of zinc from synaptic vesicles in the intact mouse brain by disruption of the ZnT3 gene. Proc Natl Acad Sci U S A 96, 1716-1721 (1999)), a protein required for Zn transport into presynaptic vesicles, which is the main pool of free Zn in the brain, were similar between genotypes (FIG. 21).

In the repetitive behavioral analysis, the vehicle-treated Shank2^{-/-} mice displayed increased jumping in their home cages compared to vehicle-treated wild-type mice, but displayed normal grooming. The behaviors were not influenced by clioquinol (FIGS. 4, 5, 23 to 26). Similarly, clioquinol did not affect repetitive behaviors in wild-type mice.

In the open field test, Shank2^{-/-} mice displayed increased locomotor activity relative to wild-type mice, as previously reported. This hyperactivity behavior did not weaken by clioquinol (FIGS. 6 and 7). Notably, Shank2^{-/-} mice spent less time in the center region of the open field arena, a measure of anxiety-like behavior. However, clioquinol had no effect on the center-region time in these mice (FIG. 27). Clioquinol had no effect on repetitive behavior, locomotor behavior, or anxiety-like behavior in wild-type mice (FIGS. 23 to 27). Together, these results signify that clioquinol improves social interaction but has no effect on social novelty recognition, repetitive behavior, hyperactivity, or anxiety-like behavior in Shank2^{-/-} mice.

### Experimental Example 2

### Evaluation of NMDAR Function in Synapses

The influence of clioquinol treatment on the NMDAR function in synapses was evaluated. As a result, clioquinol treatment restored the NMDAR function to a normal level in Shank2^{-/-} hippocampal Schaffer collateral-CA1 (SC-CA1) synapses measured by the NMDA/AMPA ratio determined by the ratio of NMDAR with respect to AMPAR-evoked excitatory postsynaptic currents (eEPSCs) (FIG. 28). However, the clioquinol treatment had no influence in wild-type synapses. In addition, clioquinol reversed the reduced tetanus-induced long-term potentiation (LTP), known to require NMDAR activity, at Shank2^{-/-} SC-CA1 synapses, but had no effect on LTP at wild-type synapses (FIG. 29). These results indicate that clioquinol restores NMDAR function at Shank2^{-/-} hippocampal SC-CA1 synapses.

Then, for NMDAR functions in Shank2^{-/-} synapses, elapsed time of clioquinol-dependent increase was measured.

In these experiments, we treated Shank2^{-/-} hippocampal slices with clioquinol for 20 minutes (in contrast to the continuous bath application for experiments described above) while monitoring NMDAR-mediated field excitatory postsynaptic potentials (NMDA-fEPSPs) before, during, and after clioquinol treatment. With clioquinol treatment, the initial slopes of NMDA-fEPSPs at Shank2^{-/-}SC-CA1 synapses gradually increased to ˜150% of baseline levels and remained elevated, a response similar to that observed in wild-type slices (FIGS. 30 and 31).

Meanwhile, in contrast to NMDA-fEPSPs, AMPAR-mediated fEPSPs (AMPA-fEPSPs) were not affected by clioquinol treatment (FIG. 34). Clioquinol also had no effect on AMPAR-related input-output ratio (AMPA-fEPSP slopes plotted against fiber volleys) or paired-pulse ratio at SC-CA1 synapses (FIGS. 35 and 36).

To further confirm the clioquinol-dependent NMDAR activation, NMDA- and AMPA-eEPSCs were simultaneously measured using patch-clamp recordings. At a holding potential of −40 mV, clioquinol increased NMDA-eEPSCs at Shank2^{-/-} SC-CA1 synapses, a result similar to that observed at wild-type synapses (FIGS. 32 and 33). The NMDAR antagonist AP5 significantly reduced NMDA-eEPSCs but not AMPA-eEPSCs, indicating that these events are NMDAR-dependent. In contrast, AMPA-eEPSCs were not affected by clioquinol treatment (FIGS. 32 and 33). Consistent with this, the NMDA/AMPA ratios derived from these currents were increased in both genotypes (FIG. 37). Taken together, these results indicate that clioquinol enhances NMDAR but not AMPAR function at Shank2^{-/-} and WT synapses.

### Experimental Example 3

### Evaluation of Whether Zn Mobilization is Required for Clioquinol-Dependent NMDAR Activation

Whether Zn mobilization is required for clioquinol-dependent NMDAR activation was evaluated. To test this, we used two different Zn chelators with much higher affinities for Zn than clioquinol: Ca-EDTA (Kd≈10⁻¹³), which is membrane-impermeable, and TPEN (Kd≈10⁻¹⁵), which is membrane-permeable. Preincubation of slices with Ca-EDTA before clioquinol treatment eliminated the clioquinol-dependent increase in NMDAR activity at Shank2^{-/-} SC-CA1 synapses, as measured by NMDA-fEPSPs (FIG. 39). In the control experiment, Ca-EDTA, by itself, did not affect NMDA-fEPSP, and this is same as previously reported (Vergnano AM, et al. Zinc dynamics and action at excitatory synapses. Neuron 82, 1101-1114 (2014), Izumi Y, Auberson Y P, Zorumski C F. Zinc modulates bidirectional hippocampal plasticity by effects on NMDA receptors. J Neurosci 26, 7181-7188 (2006)) (FIG. 38). Similar results were also obtained using TPEN (FIG. 40). Collectively, these findings suggest that clioquinol requires Zn for NMDAR activation. In addition, the absence of an effect of Ca-EDTA alone on NMDA-fEPSPs suggests that clioquinol-dependent NMDAR activation is unlikely the result of disinhibition of NMDARs by clioquinol-mediated chelation of Zn in the synaptic cleft.

Test was performed to identify the source of Zn for NMDAR activation, if the Zn-ionophoric activity of CQ is the more likely candidate mediator of NMDAR activation. One possible candidate is the Zn pool in presynaptic neurotransmitter vesicles, a major source of Zn in the brain that requires the ZnT3 transporter for its maintenance. From the result of tests of this possibility using ZnT3-deficient (ZnT3^{-/-}) mice, clioquinol had no effect on NMDAR activity in ZnT3^{-/-} SC-CA1 synapses (FIG. 40), signifying that the presynaptic Zn pool is required for the clioquinol effect. In a control experiment related to this, Zn signals were largely absent in the ZnT3^{-/-} hippocampus (FIG. 22).

The results described above were obtained in experiments performed without exogenous addition of Zn to brain slices, relying on the physiological concentrations of free Zn in the extracellular space. Previous studies have reported free Zn concentrations in the central nervous system of −20 nM, although higher concentrations might be possible (Vergnano A M, et al. Zinc dynamics and action at excitatory synapses. Neuron 82, 1101-1114 (2014)). Here, inventors of the present invention tested whether increasing the extracellular Zn concentration to 250 nM affected clioquinol-dependent NMDAR activation.

As a result, 250 nM Zn had no effect on clioquinol-dependent NMDAR activation at Shank2^{-/-} SC-CA1 synapses, as measured by NMDA-fEPSPs. This result suggests that additional Zn is not required for clioquinol-dependent NMDAR activation at Shank2^{-/-} synapses, implying that the presynaptic Zn pool under physiological conditions is sufficient for clioquinol-dependent NMDAR activation. Interestingly, 250 nM Zn significantly increased NMDA-fEPSP in the wild-type synapses (FIGS. 42 and 43).

Lastly, because clioquinol can bind Cu²⁺ (Kd≈10^{-8.9}) in addition to Zn, whether the Cu²⁺-binding activity of clioquinol also contributed to its effects on NMDAR function was tested. Application of cuprizone, a selective Cu²⁺ chelator, to hippocampal slices prior to clioquinol treatment did not inhibit the clioquinol-induced increase in NMDAR activity (FIG. 44), signifying that the Cu-binding activity of clioquinol is not important for NMDAR activation.

Taken together, these results signify that clioquinol enhances NMDAR function through its Zn-ionophoric, and NMDAR function is not enhanced through Zn-chelating or Cu²⁺-binding, activity, and mainly uses the presynaptic Zn pool for NMDAR activation.

### Experimental Example 4

### Whether Zn Mobilization Between Synapses Activates NMDAR Through Src Family Kinase

From the results of <Experimental example 1> to <Experimental example 3>, clioquinol mobilized Zn from presynaptic vesicles to synaptic clafts and postsynaptic compartments. Because Zn discharged before synapses during neuron action is known to suppress NMDAR (Vergnano A M, et al. Zinc dynamics and action at excitatory synapses. Neuron 82, 1101-1114 (2014)), it is difficult for Zn in synaptic clafts to contribute to NMDAR activation, and the inventors of the present invention identified that Ca-EDTA did not affect NMDA-fEPSP (FIG. 38).

Test was performed to identify the underlying mechanism for NMDAR activation, if postsynaptic Zn delivery is an important factor. Previous studies have shown that Zn binds to and deactivates Csk (C-terminal Src kinase), a negative regulator of Src family tyrosine kinases (SFKs), which phosphorylate and activate NMDARs (Manzerra P, et al. Zinc induces a Src family kinase-mediated up-regulation of NMDA receptor activity and excitotoxicity. Proc Natl Acad Sci U S A 98, 11055-11061 (2001), Salter M W, Kalia LV. Src kinases: a hub for NMDA receptor regulation. Nat Rev Neurosci 5, 317-328 (2004)). In experiments related to this, we identified that two independent SFK inhibitors, PP2 (FIG. 45) and SU6656 (FIG. 46), applied to hippocampal slices before clioquinol treatment removed clioquinol activation of NMDAR in Shank2^{-/-} and wild-type SC-CA1 synapses. In control experiments, PP3, an inactive PP2 analogue, failed to block the NMDAR activation (FIG. 47).

To confirm that clioquinol enhances NMDAR function through SFKs and to confirm that the subcellular site of SFK action is indeed postsynaptic, SFKs were inhibited with the Src-inhibitory peptide Src (40-58), a peptide with a sequence corresponding to Src amino acid residues 40-58 (Salter M W, Kalia L V. Src kinases: a hub for NMDA receptor regulation. Nat Rev Neurosci 5, 317-328 (2004)). Inclusion of this peptide in the patch pipette during patch-clamp recordings prevented clioquinol from increasing NMDAR activity at Shank2^{-/-} and wild-type synapses, determined by measuring NMDA-eEPSCs (FIGS. 48 and 49). In control experiments, a scrambled Src-peptide variant (sSrc 40-58) had no effect on the clioquinol-dependent increase in NMDAR activity.

In contrast to NMDARs, AMPAR function was unaffected by the Src-inhibitory peptide, as determined from simultaneous recordings of AMPA-eEPSCs at both wild-type and Shank2^{-/-} synapses (FIGS. 48 and 49). Consistent with this, the clioquinol-dependent increase in the NMDA/AMPA ratio determined from these currents was blocked by the Src-inhibitory peptide, but not by the scrambled peptide (FIGS. 50 to 53). These results signify that the clioquinol-induced increase in NMDAR function is dependent on SFKs, and imply that the subcellular site of SFK activation is postsynaptic.

### Experimental Example 5

### Evaluation of Improvement of Social Interaction in Tbr1^{+/-} Mice by Trans-Synaptic Zn Mobilization

Whether clioquinol may improve social interaction deficiency in another mouse model of ASD, in which reduced NMDAR is related to autism-like behavior. One such model is the recently developed Tbr1-haploinsufficient (Tbr1^{+/-}) mouse, which has been reported to display a reduction in social interaction that is normalized by the NMDAR agonist D-cycloserine (Huang T N, et al. Tbr1 haploinsufficiency impairs amygdalar axonal projections and results in cognitive abnormality. Nat Neurosci 17, 240-247 (2014)). Therefore, whether clioquinol may improve social interaction in the mice was evaluated.

Tbr1^{+/-} mice displayed reduced social interaction comparted to wild-type mice in the 3-chamber test. In addition, when clioquinol was acutely injected (30 mg/kg) 3 hours before the 3-chamber test, this had no effect on wild-type mice, but improved social interaction deficiencies in Tbr1^{+/-} mice (FIGS. 54 to 56, 60 and 61). However, clioquinol had no effect on social novelty recognition in Tbr1^{+/-} or wild-type mice, and this was identified based on the preference of novel stranger mouse compared to previously met mouse (FIGS. 57 to 59, 62 and 63). These results suggest that clioquinol improves social interaction deficits, but has no effect on social novelty recognition, in Tbr1^{+/-} mice.

Taken together with similar results obtained in Shank2^{-/-} mice, this signifies that clioquinol is capable of improving social interaction deficits in two independent mouse models of ASD characterized by reduced NMDAR function.

What is claimed is:

1. A pharmaceutical composition for treating autism spectrum disorders, which comprises clioquinol or pharmaceutically acceptable salt thereof.

2. The pharmaceutical composition of claim 1, wherein the clioquinol enhances postsynaptic NMDA receptor function.

3. The pharmaceutical composition of claim 1, wherein the clioquinol enhances NMDA receptor function through Zn ionophore activation.

4. The pharmaceutical composition of claim 1, wherein the clioquinol enhances NMDA receptor function through Src family tyrosine kinase.

5. The pharmaceutical composition of claim 1, wherein the clioquinol promotes Zn mobilization in synapses.

6. The pharmaceutical composition of claim 1, wherein the pharmaceutical composition improves social interaction deficiency.

7. The pharmaceutical composition of claim 1, wherein the pharmaceutical composition is administered through injection.

8. A food composition for treating autism spectrum disorders, which comprises clioquinol or pharmaceutically acceptable salt thereof.

9. The food composition of claim 8, wherein the clioquinol enhances postsynaptic NMDA receptor function.

10. The food composition of claim 8, wherein the food composition improves social interaction deficiency.
